(19) United States
(12) Patent Application Publication
(10) Pub. No.: US 2017/0073666 A1
(43) Pub. Date: Mar. 16, 2017

(54) PROCESSING OF POLYNUCLEOTIDES

(71) Applicant: BioNano Genomics, Inc., San Diego, CA (US)

(72) Inventors: William Stedman, San Diego, CA (US); Michael G Saghbini, Poway, CA (US); Han Cao, San Diego, CA (US); Alex R Hastie, San Diego, CA (US)

(21) Appl. No.: 15/123,457

(22) PCT Filed: Mar. 5, 2015
(86) PCT No.: PCT/US2015/019027

§ 371 Date: Sep. 2, 2016

Related U.S. Application Data
(60) Provisional application No. 61/949,464, filed on Mar. 7, 2014.

(51) Int. Cl. C12N 15/10

(52) U.S. Cl. CPC C12N 15/1006

(57) ABSTRACT

Methods and compositions for processing polynucleotides are provided according to some embodiments herein. In some embodiments, a sample is immobilized in a porous matrix, and non-polynucleotides are removed from the sample. In some embodiments, polynucleotides are labeled or enzymatically modified the matrix. In some embodiments, labeled or enzymatically modified polynucleotides are removed from the matrix for analysis.

### CROSS-REFERENCE TO RELATED APPLICATIONS

This application claims the benefit of U.S. Provisional Application 61/949,464, filed Mar. 7, 2014, which is hereby incorporated by reference in its entirety.

### FIELD

Embodiments herein relate generally to compositions and methods for processing of polynucleotides. More particularly, some embodiments relate generally to methods and compositions for purifying and labeling long polynucleotides from a biological sample.

### SUMMARY

In some embodiments, method of processing a sample comprising a polynucleotide is provided. The method can comprise immobilizing the sample in a thin-layer porous matrix. The method can comprise conforming the thin-layer porous matrix to a substrate. The method can comprise removing non-polynucleotide molecules from the thin-layer porous matrix conformed to the substrate while the polynucleotide remains immobilized in the thin-layer porous matrix. The method can comprise at least one of (a) labeling the polynucleotide with a first label; or (b) separating the polynucleotide from the thin-layer porous matrix. In some embodiments, the polynucleotide is labeled with the first label. In some embodiments, the polynucleotide is labeled with the first label while immobilized in the thin-layer porous matrix. In some embodiments, the polynucleotide is enzymatically labeled with the first label. In some embodiments, the polynucleotide is separated from the thin-layer porous matrix. In some embodiments, the polynucleotide is separated from the thin-layer porous matrix by at least one wash. In some embodiments, the polynucleotide is labeled with the first label and separated from the thin-layer porous matrix. In some embodiments, the polynucleotide is labeled with the first label while immobilized in the thin-layer porous matrix and subsequently separated from the thin-layer porous matrix. In some embodiments, the polynucleotide is labeled with the first label after removing non-polynucleotide molecules from the thin-layer porous matrix and before separating the polynucleotide from the matrix. In some embodiments, the polynucleotide is separated from the thin-layer porous matrix, and subsequently labeled with the first label. In some embodiments, the polynucleotide is separated from the thin-layer porous matrix by at least one wash. In some embodiments, the polynucleotide is enzymatically labeled with the first label. In some embodiments, immobilizing the sample in the thin-layer porous matrix and conforming the thin-layer porous matrix to the substrate are performed simultaneously. In some embodiments, immobilizing the sample in the thin-layer porous matrix and conforming the thin-layer porous matrix to the substrate are performed separately. In some embodiments, immobilizing the sample in the thin-layer porous matrix comprises contacting the sample with a precursor of the thin-layer porous matrix, and forming the thin-layer porous matrix is formed from the precursor comprising the sample. In some embodiments, the sample is immobilized in the thin-layer porous matrix after the thin-layer porous matrix has been formed from a precursor of the thin-layer porous matrix. In some embodiments, forming the thin-layer porous matrix comprises spreading a precursor of the thin-layer porous matrix over the substrate. In some embodiments, forming the thin-layer porous matrix comprises applying a vacuum or pressure from a gas to a precursor of the thin-layer porous matrix. In some embodiments, forming the thin-layer porous matrix comprises applying a centrifuge force to a precursor of the thin-layer porous matrix. In some embodiments, the thin-layer porous matrix is conformed to the substrate between the substrate and another entity, thereby defining at least one of a thickness, diameter, or volume of the thin-layer porous matrix. In some embodiments, conforming the thin-layer porous matrix to the substrate comprises embedding the substrate in the thin-layer porous matrix. In some embodiments, the substrate comprises a mesh. In some embodiments, the mesh comprises a plurality of opening having a diameter of 0.1 μm to 10 mm. In some embodiments, conforming the thin-layer porous matrix to the substrate comprises disposing the thin-layer porous matrix over the substrate. In some embodiments, the thin-layer porous matrix remains disposed substantially flattened over the substrate while the non-polynucleotide molecules are removed therefrom. In some embodiments, the thin-layer porous matrix is detached from the substrate, but remains in close proximity to the substrate such that the thin-layer porous matrix remains substantially flattened over the substrate. In some embodiments, the thin-layer porous matrix is maintained in close proximity to the substrate via at least one of a tether, scaffold, electromagnetic interaction, friction, or pressure so that the thin-layer porous matrix remains disposed substantially flattened over the substrate. In some embodiments, the thin-layer porous matrix is positioned between at least two posts extending from the substrate so that the thin-layer porous matrix is maintained in close proximity to the substrate. In some embodiments, the thin-layer porous matrix is positioned between the substrate and a surface such that the thin-layer porous matrix is disposed substantially flattened over the substrate. In some embodiments, the surface comprises a first mesh. In some embodiments, the substrate comprises a second mesh. In some embodiments, the first mesh comprises a plurality of openings each having a diameter of 0.1 μm to about 10 mm. In some embodiments, the second mesh comprises a plurality of openings each having a diameter of 0.1 μm to about 10 mm. In some embodiments, the thin-layer porous matrix is maintained in close proximity to the substrate via a vacuum. In some embodiments, the thin-layer porous matrix is maintained in close proximity to the substrate via pressure from a gas. In some embodiments, the thin-layer porous matrix is maintained in close proximity to the surface via a tether. In some embodiments, the tether comprises a porous material configured to maintain the thin-layer porous matrix in close proximity to the surface while allowing access to the sample immobilized in the thin layer. In some embodiments, the substrate is rigid. In some embodiments, the substrate is flexible. In some embodiments, the substrate comprises at least a portion of a slide, a container or a sheet. In some embodiments, immobilizing the sample in a thin-layer porous matrix comprises forming the thin-layer porous matrix such that the surface defines at least one side of the thin-layer porous matrix. In some embodiments, the substrate comprises a mesh. In some embodiments, the mesh comprises a plurality of openings having a diameter of 0.1 μm to about 10 mm, for example about 1 μm to about 10 mm, 10 μm to about 10 mm, 100 μm to about 10 mm, about 0.1 μm to about 1 mm, about 1 μm to about 1 mm, about 10 μm to about 1 mm, or about 100 μm to about 1 mm. In some embodiments, the thin-layer porous matrix has a thickness of about 1 to 999 micrometers. In some embodiments, the thin-layer matrix has a thickness of about 80 to 200 micrometers. In some embodiments, the thin-layer matrix is formed within a fluidic device. In some embodiments, the thin-layer matrix is formed outside of a microfluidic or nanofluidic device, and subsequently positioned within the fluidic device. In some embodiments, the thin-layer matrix is conformed to the substrate within a fluidic device. In some embodiments, the non-polynucleotide molecules are removed from the thin-layer porous matrix within a fluidic device. In some embodiments, the fluidic device is configured to control at least one of volumes, temperature, or fluidic movement during the processing. In some embodiments, the fluidic device is configured to automatically perform the processing. In some embodiments, the fluidic device comprises a microfluidic device. In some embodiments, the fluidic device comprises a nanofluidic device.

In some embodiments, a method of processing a sample comprising a polynucleotide is provided. The method can comprise immobilizing the sample in a porous matrix, in an aqueous environment. The method can comprise fragmenting the porous matrix, which comprises the immobilized sample. The method can comprise removing non-polynucleotide molecules from the porous matrix while the polynucleotide remains in the porous matrix. The method can comprise separating the polynucleotide from the porous matrix. In some embodiments, non-polynucleotide molecules are removed from the porous matrix after fragmenting the porous matrix. In some embodiments, non-polynucleotide molecules are removed from the porous matrix prior to fragmenting the porous matrix. In some embodiments, the method further comprises removing traces of non-polynucleotide molecules from the porous matrix after fragmenting the matrix, wherein polynucleotide molecules remain in the porous matrix while the traces of non-polynucleotide molecules are removed. In some embodiments, the method further comprises labeling the polynucleotide with a first label after removing non-polynucleotide molecules from the porous matrix and before separating the polynucleotide from the matrix. In some embodiments, the sample is immobilized in the porous matrix within a fluidic device. In some embodiments, the porous matrix is formed outside of a microfluidic or nanofluidic device, and subsequently positioned within the fluidic device. In some embodiments, the porous matrix is conformed to the substrate within a fluidic device. In some embodiments, the non-polynucleotide molecules are removed from the porous matrix within a fluidic device. In some embodiments, the fluidic device is configured to control at least one of volumes, temperature, or fluidic movement during the processing. In some embodiments, the fluidic device is configured to automatically perform the processing. In some embodiments, the fluidic device comprises a microfluidic device. In some embodiments, the fluidic device comprises a nanofluidic device.

In some embodiments, for any of the above methods, the polynucleotide comprises at least about 200 kilobases, for example, at least about 200 kb, 250 kb, 300 kb, 350 kb, 400 kb, 450 kb, 500 kb, 550 kb, 600 kb, 650 kb, 700 kb, 750 km 850 kb, 950 kb or 1000 kb, including ranges between any two of the listed values. In some embodiments, for any of the above methods, the polynucleotide comprises at least about 1 megabase. In some embodiments, for any of the above methods, the sample comprises at least one of a cell suspension, a nuclei suspension, an organelle suspension, a cell homogenate, a tissue homogenate, a whole organism homogenate, and a biological fluid. In some embodiments, for any of the above methods, the sample comprises a whole cell. In some embodiments, for any of the above methods, the polynucleotide comprises single-stranded DNA, single-stranded RNA, double-stranded DNA, or double-stranded RNA. In some embodiments, for any of the above methods, the porous matrix or thin-layer porous matrix comprises a synthetic polymer, a naturally occurring polymer, or a combination thereof. In some embodiments, for any of the above methods, the porous matrix or thin-layer porous matrix comprises a polysaccharide-based matrix. In some embodiments, for any of the above methods, the porous matrix or thin-layer porous matrix comprises an agarose matrix, a polyacrylamide matrix, a gelatin matrix, a collagen matrix, a fibrin matrix, a chitosan matrix, an alginate matrix, a hyaluronic acid matrix, or any combination thereof. In some embodiments, for any of the above methods, the porous matrix or thin-layer porous matrix comprises an agarose matrix. In some embodiments, for any of the above methods, the porous matrix or thin-layer porous matrix comprises a silane group, a positively charged group, a negatively charged group, a zwitterionic group, a polar group, a hydrophilic group, a hydrophobic group, or any combination thereof. In some embodiments, for any of the above methods, the porous matrix or thin-layer porous matrix comprises an aqueous environment. In some embodiments, for any of the above methods, the porous matrix or thin-layer porous matrix is disposed in an aqueous solution. In some embodiments, for any of the above methods, non-polynucleotide molecules comprise at least one of a protein, a lipid, a carbohydrate, an organelle, and cellular debris. In some embodiments, for any of the above methods, removing non-polynucleotide molecules comprises contacting the porous matrix or thin-layer porous matrix with a proteinase, an elastase, a collagenase, a lipase, a carbohydratase, a pectinase, a pectolyase, an amylase, an RNase, a hyaluronidases, a chitinase, a gluculase, a lyticase, a zymolyase, a lysozyme, a labiase, an achromopeptidase, or a combination thereof. In some embodiments, for any of the above methods, removing non-polynucleotide molecules comprises contacting the porous matrix or thin-layer porous matrix with a proteinase. In some embodiments, for any of the above methods, removing non-polynucleotide molecules comprises contacting the porous matrix or thin-layer porous matrix with a detergent, a chaotrope, a buffer, a chelator, an organic solvent, a polymer, an alcohol a salt, an acid, a base, a reducing agent, or a combination thereof. In some embodiments, for any of the above methods, the polymer comprises one of polyethylene glycol, polyvinypyrrolidone, polyvinyl alcohol, or ethylene glycol. In some embodiments, for any of the above methods, the organic solvent is miscible in an aqueous based solution. In some embodiments, for any of the above methods, removing non-polynucleotide molecules comprises applying an electric field to remove at least some non-polynucleotide molecules. In some embodiments, for any of the above methods, the method further comprises in-matrix nuclei enrichment prior to removing non-polynucleotide molecules. In some embodiments, for any of the above methods, the labeling comprises non-site-specific labeling. In some embodiments, for any of the above methods, the labeling comprises site-specific labeling. In some embodiments, for any of the above methods, the labeling comprises contacting the polynucleotide with a dye or stain. In some embodiments, for any of the above methods, the labeling comprises non-optical labeling. In some embodiments, for any of the above methods, the polynucleotide is double-stranded, and site-specific labeling comprises nicking the polynucleotide at a first sequence motif, thereby forming at least one nick, wherein the DNA remains double-stranded adjacent to the at least one nick; and labeling the at least one nick with the first label. In some embodiments, for any of the above methods, the polynucleotide is immobilized in the matrix when nicked. In some embodiments, for any of the above methods, the site-specific labeling comprises incorporating at least one nucleotide into the at least one nick. In some embodiments, for any of the above methods, at least one nucleotide comprises a reversible terminator. In some embodiments, for any of the above methods, the at least one nucleotide comprises the first label. In some embodiments, for any of the above methods, the methods further comprises nicking the polynucleotide at a second sequence motif, thereby forming at least one second nick, wherein the DNA remains double-stranded adjacent to the at least one second nick; and labeling the at least one second nick with a second label, wherein the first label and the second label are the same or different. In some embodiments, for any of the above methods, the labeling comprises transferring the label to the polynucleotide by a first methyltransferase. In some embodiments, for any of the above methods, the site-specific labeling comprises transferring the first label to a first sequence motif by a first methyltransferase. In some embodiments, for any of the above methods, the site-specific labeling comprises transferring a first reactive group to the first sequence motif; and coupling the first label to the first reactive group. In some embodiments, for any of the above methods, the method further comprises transferring a second label to a second sequence motif by a second methyltransferase, wherein the second sequence motif is different from the first sequence motif, and wherein the second label is the same or different from the first label. In some embodiments, for any of the above methods, site-specific labeling comprises contacting a first sequence motif of the polynucleotide immobilized in the matrix with a first binding moiety that binds specifically to the first sequence motif. In some embodiments, for any of the above methods, the first binding moiety comprises one of a triple helix oligonucleotide, a peptide, a nucleic acid, a polyamide, a zinc finger DNA binding domain, a transcription activator like (TAL) effector DNA binding domain, a transcription factor DNA binding domain, a restriction enzyme DNA binding domain, an antibody, or any combination thereof. In some embodiments, for any of the above methods, at least one of the first label or the second label is selected from the group consisting of a fluorophore, a quantum dot, or a non-optical label. In some embodiments, for any of the above methods, the method further comprises labeling the polynucleotide with a non-sequence-specific label, wherein the non-sequence specific label is different from the first and second labels. In some embodiments, for any of the above methods, separating comprises at least one of melting the porous matrix, digesting the porous matrix, degrading the porous matrix, solubilizing the porous matrix, electroelution, spinning through a sieve, blotting onto a membrane, dialysis step, or a combination thereof. In some embodiments, for any of the above methods, separating comprises adding a solvent to a mixture comprising the polynucleotide and at least one component of the matrix. In some embodiments, for any of the above methods, the method further comprises detecting a pattern of site-specific labeling characteristic of the polynucleotide. In some embodiments, for any of the above methods, detecting comprises linearizing the polynucleotide in a fluidic channel. In some embodiments, for any of the above methods, the method further comprises comparing a pattern of the first label, second label or any combination thereof to a pattern of labels on a reference DNA. In some embodiments, for any of the above methods, the method further comprises assembling a plurality of patterns based on overlapping patterns of site-specific labeling, thereby constructing a polynucleotide map.

In some embodiments, a polynucleotide preparation is provided. The preparation can comprise a thin-layer porous matrix conformed to a substrate. The preparation can comprise a polynucleotide immobilized in the porous matrix, in which the polynucleotide is substantially isolated from non-polynucleotide cellular components, and in which the polynucleotide has been site-specifically labeled or enzymatically modified while in the matrix. In some embodiments, the thin-layer porous matrix is disposed substantially flat over the substrate. In some embodiments, the substrate is embedded in the thin-layer porous matrix. In some embodiments, the substrate is positioned on a first side of the thin-layer porous matrix, and wherein a surface is position on a second side of the thin-layer porous matrix. In some embodiments, the substrate comprises a mesh. In some embodiments, the mesh comprises a plurality of openings, each having a diameter of 0.1 μm to 10 mm. In some embodiments, the surface comprises a second mesh. In some embodiments, the second mesh comprises a plurality of openings, each having a diameter of 0.1 μm to 10 mm. In some embodiments, the polynucleotide was separated from cellular components while in the matrix. In some embodiments, the polynucleotide was labeled prior to separation from cellular components. In some embodiments, the polynucleotide was labeled after separation from cellular components. In some embodiments, the polynucleotide comprises at least about 200 kilobases, for example, at least about 200 kb, 250 kb, 300 kb, 350 kb, 400 kb, 450 kb, 500 kb, 550 kb, 600 kb, 650 kb, 700 kb, 750 km 850 kb, 950 kb or 1000 kb, including ranges between any two of the listed values. In some embodiments, the polynucleotide comprises at least about 1 megabase. In some embodiments, the polynucleotide comprises single-stranded DNA, single-stranded RNA, double-stranded DNA, or double-stranded RNA. In some embodiments, the thin-layer porous matrix comprises a synthetic polymer, a naturally occurring polymer, or a combination thereof. In some embodiments, the thin-layer porous matrix comprises a polyacrylamide matrix, a gelatin matrix, a collagen matrix, a fibrin matrix, a chitosan matrix, an alginate matrix, a hyaluronic acid matrix, or any combination thereof. In some embodiments, the thin-layer porous matrix comprises an agarose matrix. In some embodiments, the thin-layer porous matrix comprises a polysaccharide-based matrix. In some embodiments, the porous matrix comprises a silane, a positively charged group, a negatively charged group, a zwitterionic group, a polar group, a hydrophilic group, a hydrophobic group, or any combination thereof. In some embodiments, the thin-layer porous matrix is disposed over the surface in an extended configuration. In some embodiments, the thin-layer matrix has a thickness of about 1 to 999 micrometers. In some embodiments, the thin-layer porous matrix has a thickness of about 80 to 200 micrometers. In some embodiments, the thin-layer porous matrix is immobilized on the surface. In some embodiments, the thin-layer porous matrix is detached from the surface, but remains in close proximity to the surface such that the layer remains substantially extended throughout the processing. In some embodiments, the surface is rigid. In some embodiments, the surface is flexible. In some embodiments, the surface is that of a slide, a container or a sheet. In some embodiments, the thin-layer porous matrix is substantially free of non-polynucleotide cellular components. In some embodiments, the non-polynucleotide cellular components comprise at least one of proteins, lipids, carbohydrates, organelles, and cellular debris. In some embodiments, the site-specific labeling or enzymatic modification comprises labeling with at least a first label associated with a first sequence motif. In some embodiments, the site-specific labeling or enzymatic modification further comprises labeling with a second label associated with a second sequence motif, wherein the second label is the same as or different from the first label. In some embodiments, the site-specific labeling comprises labeling with at least a labeled oligonucleotide incorporated into a nick in a double-stranded DNA or RNA. In some embodiments, the preparation further comprises at least one binding moiety bound to the first motif, wherein the binding moiety comprises at least one of a triple helix oligo, a peptide nucleic acid, a polyamide, a zinc finger DNA binding domain, a transcription activator like (TAL) effector DNA binding domain, a transcription factor DNA binding domain, a restriction enzyme DNA binding domain, an antibody, or a combination thereof. In some embodiments, the site specific labeling comprise labeling with a label selected from the group consisting of a fluorophore, a quantum dot, and a non-optical label.

According to some embodiments herein, a method of processing a sample is provided. The method can comprise immobilizing the sample in a thin-layer porous matrix disposed over a substrate. The method can comprise processing the sample trapped in the substrate-associated layer to remove undesired components while at least one desired component remains immobilized in the sample. The method can comprise separating the at least one desired component from the porous matrix. The method can comprise characterizing the at least one desired component. In some embodiments, the desired component comprises at least one of a nucleic acid, a protein, a carbohydrate, a lipid, a polysaccharide, a metabolite, a small molecule, an antibody, or a combination thereof. In some embodiments, the desired component is a DNA, and wherein the characterizing comprises determining, a concentration, a quality metric, a physical map, a sequence content, an epigenetic information, a SNP, a haplotype, an RFLP, a sizing, a copy number variants, or any combination of these. In some embodiments, the desired component is an RNA, and wherein the characterizing comprises determining, a concentration, a quality metric, a sequence content, an expression level, a stability, a splicing event, or any combination thereof. In some embodiments, the desired component is a protein, and wherein the characterizing comprises determining, a concentration, a purity, a sequence content, a structural property, an antibody reactivity, an enzymatic activity, an inhibitory activity, a post translational modifications, a toxic effect, or any combination of these. In some embodiments, the method further comprises labeling the polynucleotide or covalently modifying the polynucleotide, while the polynucleotide is in the matrix.

In some embodiments, a system for processing a sample containing at least one polynucleotide is provided. The system can comprise a porous matrix configured to be formed into a thin-layer porous matrix comprising the sample. The system can comprise a substrate for forming the thin-layer porous matrix. The system can comprise a means for maintaining the thin-layer porous-matrix conformed to substrate. In some embodiments, the system further comprises a means for forming a well around the thin-layer porous-matrix substantially disposed over the substrate. In some embodiments, the system further comprises a means for maintaining the thin-layer porous matrix at a desired temperature. In some embodiments, the system further comprises a purification reagent for removing a sample component other than the at least one polynucleotide, and a first labeling reagent for labeling a sequence motif of the at least one polynucleotide with a first label, and a separation reagent for separating the labeled polynucleotide from the thin-layer porous-matrix, wherein patterns of sequence motif labeling of the separated polynucleotide can be characterized. In some embodiments, the substrate comprises a first mesh, and the means for maintaining the thin-layer porous matrix conformed to the substrate comprises a second mesh. In some embodiments, the first mesh and the second mesh each comprise a plurality of openings, wherein each opening has a diameter of 0.1 μm to 10 mm. In some embodiments, the system comprises a fluidic system. In some embodiments, the system is configured to automatically form the thin-layer porous matrix. In some embodiments, the system is configured to receive a pre-formed thin-layer porous matrix. In some embodiments, the system is configured to automatically separate the labeled polynucleotide from the thin-layer porous matrix. In some embodiments, the system comprises a microfluidic system. In some embodiments, the porous matrix is in fluid communication with a nanochannel.

In some embodiments, a kit for forming a thin-layer porous matrix is provided. The kits can comprise a substrate; and a well-forming apparatus comprising one or more openings configured to define on or more surfaces perpendicular or substantially perpendicular to the substrate when placed against the substrate. In some embodiments, the kit further comprises a thin-layer porous matrix precursor. In some embodiments, the well-forming apparatus comprises a sealing member configured to form a seal against the substrate. In some embodiments, the kit further comprises a compression plate configured to immobilize the well-forming apparatus against the substrate. In some embodiments, the kit further comprises the kit further comprising a heating member configured to heat the substrate and the well-forming apparatus. In some embodiments, the kit further comprises a mesh. In some embodiments, the substrate comprises a PTFE coating that forms a plurality of features configured to maintain a thin-layer porous matrix disposed over the substrate. In some embodiments, the kit further comprises a fluidic device.

In some embodiments, for any of the above methods, the substrate comprises a loose or tight mesh, thereby forming the precursor material into a thin layer intercalated between or on one or more surfaces of fibers of the mesh, thereby immobilizing the sample in a thin-layer porous matrix. In some embodiments, for any of the above methods, the thin-layer porous matrix is immobilized as a thin layer intercalated between on one or more surfaces of fibers of a mesh or on one or more surfaces of the fibers of the mesh, so as to facilitate washing or labeling of polynucleotides from a first and a second surface of the mesh. In some embodiments, for any of the above methods, the substrate comprises features, thereby forming the precursor material into a thin layer intercalated between features, thereby immobilizing the sample in a thin-layer porous matrix. In some embodiments, the features comprise posts. In some embodiments, for any of the above methods, the thin-layer porous matrix is formed by contact with a change in air pressure such as compressed air or vacuum, thereby forming the precursor material into a thin layer, thereby immobilizing the sample in a thin-layer porous matrix. In some embodiments, contact with a change in air pressure comprises compressed air or a vacuum. In some embodiments, for any of the above methods, the thin-layer porous matrix is formed by contact with centrifugal force, such as that from a centrifuge, thereby forming a precursor of the thin-layer porous matrix into a thin layer, thereby immobilizing the sample in a thin-layer porous matrix. In some embodiments, contact with centrifugal force comprises force from a centrifuge.

### BRIEF DESCRIPTION OF THE DRAWINGS

FIG. 1 is a flow diagram illustrating a method of processing a sample comprising a polynucleotide according to some embodiments herein.

FIG. 2 is a flow diagram illustrating a method of processing a sample comprising a polynucleotide according to some embodiments herein.

FIGS. 3A and 3B are photographs illustrating a thin-layer porous matrix on a slide according to some embodiments herein. As shown in FIG. 3A, 20u1 agarose-E. coli mixture was deposited on a slide and spread by sandwiching with another slide in the presence of 80 μm spacers. As shown in FIG. 3B, an agarose-mammalian cultured cells in a thin-layer porous matrix was produced in a well, 14 mm in diameter 100 μm in height, defined by PTFE coating on the slide (black color).

FIGS. 4A and 4B are photographs illustrating agarose-E. coli mixtures deposited in culture plates according to some embodiments herein. As shown in FIG. 3A, 20 ul agarose-E. coli mixture was deposited in a well of a 6 well culture plate. As shown in FIG. 3B, 900 ul agarose-E. coli mixture was deposited in a 10 cm culture plate. In each of FIGS. 3A and 3B, the agarose-E. coli mixture was spread with a pipet tip to achieve a thin layer attached to the bottom of its container. A nylon mesh was added to keep the layer tethered to the surface.

FIG. 5A is a photograph illustrating labeled DNA molecules extended in nanochannels (Irys™ platform, BioNano Genomics) according to some embodiments herein. Backbone (non-sequence-specific) staining is shown in (I). A red (site-specific) labeling pattern for the two molecule in (I) is shown in (II) while a green (site-specific) labeling pattern is shown in (III).

FIG. 5B is a table illustrating labeling metrics for the labeled DNA of FIG. 5A. Thin-layer porous matrix DNA purification was performed on a slide followed by sequence-specific labeling in-matrix with one color or successive labeling with two colors (G: green label; R: red label; FP: False Positive; FN: False Negative).

FIG. 6 is a table illustrating labeling metrics for tethered thin layer DNA purification in a well followed by sequence-specific labeling in thin layer, performed according to some embodiments herein (FP: False Positive; FN: False Negative).

FIG. 7 is a table illustrating labeling metrics for microlayer/thin layer DNA purification followed by sequence-specific labeling in solution, performed according to some embodiments herein (FP: False Positive; FN: False Negative).

FIG. 8 is a table illustrating labeling metrics for large scale thin layer DNA purification in plate followed by sequence-specific labeling in solution, performed according to some embodiments herein (FP: False Positive; FN: False Negative).

FIG. 9 is a table illustrating labeling metrics for plug/porous units DNA purification followed by sequence specific labeling in porous units, performed according to some embodiments herein (FP: False Positive; FN: False Negative).

FIGS. 10A, 10B, 10C, and 10D are photographs illustrating a sample processing device according to some embodiments herein. FIG. 10A illustrates a metallic base 10 for holding a slide that fits a heat block for temperature control. FIG. 10B illustrates a slide comprising a thin-layer porous matrix tethered with a mesh 12 placed on the metallic base 10. FIG. 10C illustrates a well forming unit 14, including a reaction well 16 and o ring 18. FIG. 10 D illustrates the well forming unit 14 assembled on slide comprising the thin-layer porous matrix tethered with a nylon mesh 12 and showing the reaction well 16. Also shown is a lid 20 to close reaction well.

FIG. 11 is a table illustrating labeling metrics for thin-layer porous matrix DNA purification and sequence specific labeling in the slide processing device illustrated in FIG. 10 (FP: False Positive; FN: False Negative).

FIG. 12A is a table illustrating de novo mapping results for human genomic material purified using a thin-layer porous matrix.

FIG. 12B is a graphic illustrating the assembled genomic map from the de novo mapping described in FIG. 12A.

FIGS. 13A, 13B, and 13C are photographs illustrating a sample processing device according to some embodiments herein. FIG. 13A illustrates a side view of the device. FIG. 13B illustrates a top view of the device (in the absence of a cover). FIG. 13C illustrates a top view of the device in the presence of a cover.

FIG. 14 is a photograph illustrating a sample processing device #2 according to some embodiments herein. FIG. 14 illustrates a metallic base 140 for holding a slide that fits a heated oven for temperature control.

FIG. 15 is a photograph illustrating a sample processing device #2 according to some embodiments herein. FIG. 15 illustrates a slide comprising a thin-layer porous matrix spread within a polyfluortetraethylene (PTFE) ring 143 tethered with a mesh 144 placed on the metallic base 145.

FIGS. 16A-G is a series of photographs illustrating a sample processing device #2 according to some embodiments herein. FIG. 16A illustrates a well forming unit 165. FIG. 16B illustrates a well forming unit 165, including a reaction well 167 and o-ring 166. FIG. 16C illustrates a wave washer 168. FIG. 16D illustrates the well forming unit 165 positioned on slide and comprising the thin-layer porous matrix tethered with a nylon mesh 164 and showing the wave washer 168 positioned over each reaction well 167. FIG. 16E illustrates a metallic compression plate 169. FIG. 16F illustrates the well forming unit 165 assembled on slide comprising the thin-layer porous matrix tethered with a nylon mesh 164 and showing the compression plate 169 positioned over well forming unit 165. FIG. 16G illustrates the positioning of an adhesive sealing film 170 to create top air seal.

FIGS. 17A and 17B are schematic diagrams showing features of PTFE coating rings on slides according to some embodiments herein. The PTFE features can comprise retaining posts for a thin-layer porous matrix. FIG. 17A illustrates PTFE features 171 around an inside diameter of a PTFE ring 163 coated on a slide 162 for holding a thin-layer porous matrix in place during processing. FIG. 17B illustrates PTFE features 171 positioned uniformly over entire well inside PTFE ring 173 coated on a slide 172 for holding thin-layer porous matrix in place during processing.

FIGS. 18A and 18B are photographs illustrating thin-layer porous matrices formed in accordance with some embodiments herein. FIG. 18A illustrates a thin-layer porous matrix on a slide. FIG. 18A illustrates the formation of a thin-layer porous matrix 172 on a slide 162 after compressed air was applied to precursor material. FIG. 18B illustrates a thin-layer porous matrix on a porous mesh substrate. FIG. 18B illustrates the formation of a thin-layer porous matrix 172 on mesh 164 after precursor material was compressed between two slides.

FIG. 19 is a table illustrating reduction in time to complete a thin-layer porous matrix (e.g. microlayer) protocol in accordance with some embodiments herein protocol with metrics for microlayer/thin layer DNA purification followed by sequence-specific labeling in matrix (row 1) or in solution (row 2), performed according to some embodiments herein using sample preparation device #2 (FP: False Positive; FN: False Negative).

FIG. 20 is a table illustrating DNA yield and DNA concentration after thin layer porous matrix (e.g. microlayer) DNA purification in accordance with some embodiments herein followed by sequence-specific labeling in matrix (row 1) or in solution (row 2), performed according to some embodiments herein using sample preparation device #2.

FIG. 21 is a table illustrating the human genome depth (x coverage) required to achieve ≧1 Mb n50 contig size for plug methods vs. thin-layer porous matrix (e.g. microlayer) methods accordance with some embodiments herein on slide processing, performed according to some embodiments herein using sample preparation device #2.

FIG. 22 is a table illustrating the inversion (genome structural variations) detection sensitivity of thin-layer porous matrix (e.g. microlayer) methods accordance with some embodiments herein relative to plug method of DNA purification and labeling for a set of 187 know inversions, collected from the Database of Genomic Variants (DGV), that were incorporated into hg19.

FIG. 23 is a table illustrating the increased n50 contig size (Mb) at higher genome depth of thin-layer porous matrix (e.g. microlayer) methods accordance with some embodiments herein relative to plug method of DNA purification and labeling.

FIG. 24 is a table illustrating the decreased fragile site distance (bp) of thin-layer porous matrix (e.g. microlayer) methods in accordance with some embodiments herein relative to plug method of DNA purification and labeling.

FIG. 25 is a table illustrating the increased DNA size (kb) of thin-layer porous matrix (e.g. microlayer) methods in accordance with some embodiments herein relative to plug method of DNA purification and labeling. It is noted that while plugs can purify DNA of up to 350 kb to 400 kb, thin-layer porous matrix approaches in accordance with some embodiments herein can purify DNA of at least 1000 kb (in addition to DNA's less than 1000 kb).

### DETAILED DESCRIPTION

Genome mapping at the single molecule level can involve purification and labeling of polynucleotides, some of which contain at least a megabase of material. According to some embodiments herein, methods and structures for purification and optionally labeling polynucleotides are provided. In some embodiments, the polynucleotide is immobilized in a porous matrix. In some embodiments, the porous matrix has a high surface area relative to its volume. A high surface area can facilitate removal of non-polynucleotide molecules and other manipulations while the polynucleotide remains immobilized in the porous matrix. In some embodiments, the polynucleotide is labeled while immobilized in the porous matrix. In some embodiments, the polynucleotide is removed from the matrix and then labeled.

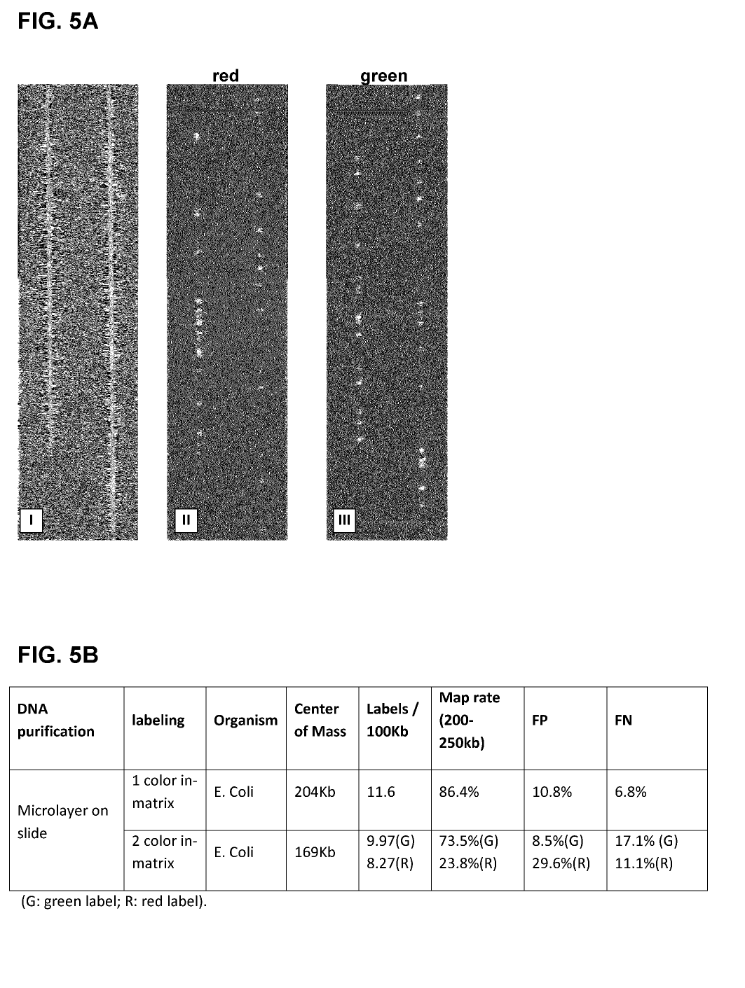

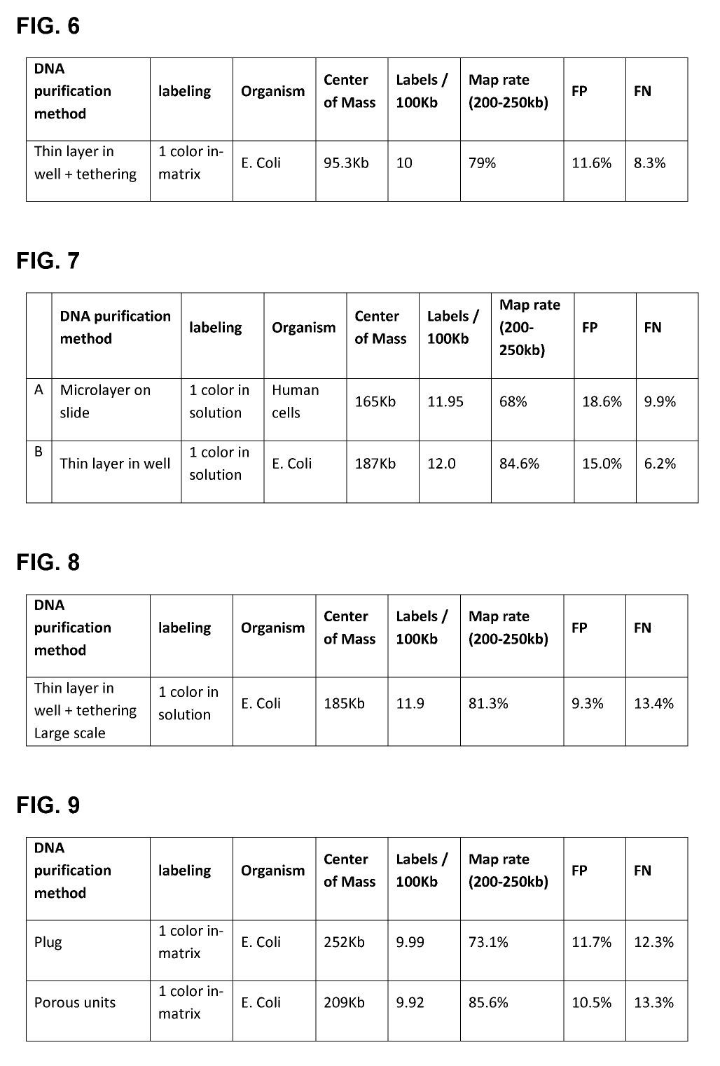

Methods and compositions for processing polynucleotides according to some embodiments herein can yield high purity megabase-containing polynucleotide molecules, and can facilitate labeling and removal of non-polynucleotide molecules. Traditionally, processing large polynucleotide molecules has included embedding and purifying biological samples in agarose plugs. However, typical mechanical manipulations for generating purified polynucleotides in such plugs can restrict the plug's accessible surface area, and can make the plug containing the polynucleotide a poor candidate for in-matrix reactions. While biological samples can be embedded in in agarose microbeads or fibers to increase surface area for in-matrix reactions, such configurations can limit mechanical processing for purification and/or sequential enzymatic processing. Additionally, microbeads can be difficult to produce and handle. For example, microbeads can stick to the sides of tubes and inside pipet tips and become readily transparent and difficult to visualize. Furthermore, methods of making the microbeads can yield inconsistent results. Agarose fibers, also known as “agarose worms” can pose similar challenges as microbeads. It is appreciated herein that processing of polynucleotides according to some embodiments herein can purify polynucleotides (including large polynucleotides such as megabase-containing polynucleotides) from sample contaminants, and facilitate high-efficiency polynucleotide manipulations while the polynucleotide is immobilized in an accessible format in a porous matrix. As shown in Examples 1-6 (see also labeling metrics of FIG. 5B, FIGS. 6-9, and FIG. 11), embodiments herein can yield purified polynucleotides with high rates of labeling, and low false positive (FP) and low false negative (FN) rates. The polynucleotides can be subsequently recovered and analyzed.

### Porous Matrices

According to some embodiments, a porous matrix is provided. The porous matrix can comprise pores to permit the movement of molecules such as labels and non-polynucleotide molecules (e.g. molecules being removed from a sample) in, out, and within the matrix. In some embodiments, a porous matrix is formed from a precursor material. For example, a liquid agarose solution can form a matrix upon cooling. Accordingly, in some embodiments, a polynucleotide is embedded in a porous matrix by contacting the polynucleotide with the precursor material, and then forming the matrix so that the polynucleotide is embedded therein.

In some embodiments, the porous matrix comprises a synthetic polymer, a naturally-occurring polymer, or a combination of the two. In some embodiments, the porous matrix comprises an agarose matrix, a polyacrylamide matrix, a gelatin matrix, a collagen matrix, a fibrin matrix, a chitosan matrix, an alginate matrix, a hyaluronic acid matrix, or a combination of two or more of the listed items, for example two, three, four, five, six, seven, or eight of the listed items. In some embodiments, a combination of precursors of two or more of the listed materials is combined, and formed into a porous matrix. In some embodiments, porous matrices formed of two or more of the listed materials are formed and then combined. In some embodiments, the porous matrix is an agarose matrix. In some embodiments, the porous matrix is a polysaccharide-based matrix. As some samples, for example nucleic acids, can be soluble in aqueous environments, in some embodiments, the porous matrix comprises an aqueous environment. In some embodiments, the matrix itself is disposed in an aqueous environment, for example an aqueous buffer.

It can be useful for a porous matrix to include one or more functional groups, depending on the desired function of the porous matrix. For example, without being limited by any particular theory, removal of hydrophobic materials from the matrix can be facilitated by the inclusion of hydrophilic functional groups in the matrix. For example, without being limited by any particular theory, immobilization of polynucleotides in the matrix can be facilitated by positively charged functional groups in the matrix. As such, in some embodiments, the porous matrix comprises a silane, a positively charged group, a negatively charged group, a zwitterionic group, a polar group, a hydrophilic group, a hydrophobic group, or a combination of two or more of the listed items, for example two, three, four, five, six, or seven of the listed items

### Thin-Layer Porous Matrices

As used herein, a thin-layer porous matrix refers to porous matrix material that has a thickness that is less than either its width or length, in which the thickness is no greater than 999 micrometers. In some embodiments, the thin-layer porous matrix has a thickness of no more than 999 micrometers, for example about 1 micrometer, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 960, 970, 980, 990, 991, 992, 993, 994, 995, 996, 997, 998, or 999 micrometers, including ranges between any two of the listed values. In some embodiments, the thin layer has a thickness of about 1 micrometer to about 999 micrometers, 1 micrometer to 800 micrometers, 1 micrometer to 600 micrometers, 1 micrometer to 400 micrometers, 1 micrometer to 200 micrometers, 1 micrometer to 150 micrometers, 1 micrometer to 100 micrometers, 10 micrometers to 800 micrometers, 10 micrometers to 600 micrometers, 10 micrometers to 400 micrometers, 10 micrometers to 200 micrometers, 10 micrometer to 150 micrometers, 10 micrometers to 100 micrometers, 20 micrometers to 800 micrometers, 20 micrometers to 600 micrometers, 20 micrometers to 400 micrometers, 20 micrometers to 200 micrometers, 20 micrometers to 100 micrometers, 20 micrometers to 150 micrometers, 50 micrometers to 800 micrometers, 50 micrometers to 600 micrometers, 50 micrometers to 400 micrometers, 50 micrometers to 200 micrometers, 50 micrometers to 100 micrometers, 50 micrometers to 150 micrometers, 100 micrometers to 800 micrometers, 100 micrometers to 600 micrometers, 100 micrometers to 400 micrometers, or 100 micrometers to 200 micrometers.

In some embodiments, a thin-layer porous matrix is formed from a precursor material. In some embodiments, the porous matrix is formed from a precursor material that contains a biological sample comprising at least one polynucleotide. For example, a biological sample can be added to a liquid precursor of the porous matrix, so that when the liquid precursor is formed into the porous matrix, the biological sample can be immobilized therein.

In some embodiments, the thin-layer porous matrix does not contain the biological sample or polynucleotides at the time the matrix is formed. The biological sample or polynucleotides can be added to the thin-layer porous matrix, for example by applying an electric field. In some embodiments, the biological sample or polynucleotide is added concurrently with the formation of the porous matrix.

In some embodiments, the porous matrix is associated with a substrate. A wide variety of substrates can be used in conjunction with embodiments herein. In some embodiments, the substrate is rigid. In some embodiments, the substrate is flexible. In some embodiments, the substrate comprises a surface of a slide, a container or a sheet. In some embodiments, forming the thin-layer porous matrix is formed such that the substrate defines at least one side of the thin-layer porous matrix. In some embodiments, thin-layer porous matrix is formed between the substrate and another surface or set of surfaces, thus defining at least one of a thickness, diameter, or volume of the thin-layer porous matrix. For example, an matrix precursor liquid such as agarose can be cooled in a mold to form a thin-layer porous matrix. For example, a thicker porous matrix can be mechanically or chemically manipulated, for example by shaving, cutting, grinding, or dissolving a portion of the matrix away to form it into a thin layer. In some embodiments, a precursor material is positioned on a substrateand spread into a thin layer, which forms the porous matrix. In some embodiments, the substrate is embedded in the thin-layer porous matrix. Optionally, the substrate comprises a mesh comprising a plurality of nanometer- or micrometer-scale openings as described herein.

A thin-layer porous matrix that “conforms” to a substrate refers to a major surface (i.e. one of the two surfaces with the greatest surface area) of the thin-layer porous matrix positioned parallel or substantially parallel to the major surface of the substrate. The major surface of the thin-layer porous matrix is considered substantially parallel to the major surface of the substrate if, when viewed in profile view (so as to consolidate representations of both diameters into a single plane), the longest diameter of the major surface of the thin-layer porous matrix would be parallel to the longest diameter of the major surface of the substrate or would intersect the major surface of the substrate at an angle of less than 15°. In some embodiments, the thin-layer porous matrix is conformed to the substrate so that the substrate in embedded in the thin-layer porous matrix. In some embodiments, the thin-layer porous matrix is conformed to the substrate so that it is disposed over the substrate in a substantially flat configuration. As used herein, “substantially flat” and variations of this root term refers to the thin-layer porous matrix having no two non-adjacent edges touching, and a longest diameter and diameter perpendicular thereto that are each at least 80% of the longest diameter and diameter perpendicular thereto, respectively when the thin-layer porous matrix is spread in a completely flat, but unstrained configuration. In some embodiments, thin-layer porous matrix remains disposed over the substrate in a substantially flat configuration throughout the removal of non-polynucleotides from the porous matrix. In some embodiments, thin-layer porous matrix remains disposed over the substrate in a substantially flat configuration while the polynucleotide is labeled. In some embodiments, the thin-layer porous matrix is immobilized on the substrate. In some embodiments, the thin-layer porous matrix is covalently bound to the substrate. In some embodiments, the thin-layer porous matrix is but remains in close proximity to the surface such that the layer remains substantially extended substantially flat throughout the processing. In some embodiments, the thin-layer porous matrix is in a substantially flat configuration, and no point on the longest diameter of the thin-layer porous matrix is more than 100 micrometers from the substrate, for example no more than 100 micrometers, 99, 90, 80, 70, 60, 50, 40, 30, 20, 10, 9, 8, 7, 5, 6, 5, 4, 3, 2, or 1 micrometer from the substrate.

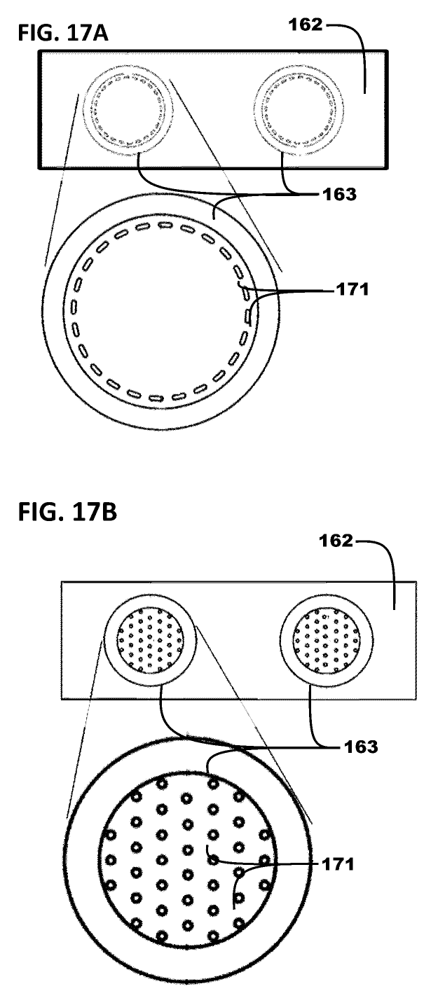

In some embodiments, thin-layer porous matrix is maintained in a substantially flat configuration. The thin-layer porous matrix can be maintained in a substantially flat configuration throughout processing of the sample. In some embodiments, the thin-layer porous matrix is tethered to the substrate, for example via a mesh or webbing configured to maintain the thin-layer porous matrix in a substantially flat configuration and in close proximity to the substrate. In some embodiments, the thin-layer porous matrix is maintained in a substantially flat configuration and in close proximity to the substrate via at least one of a pressure, friction, or electromagnetic forces. In some embodiments, the thin-layer porous matrix is positioned between two or more features protruding from the substrate, such as posts, for example, at least 2, 3, 4, 5, 6, 7, 8, 9, 10, 15, 20, 25, 30, 40, 50, 60, 70, 80, 90, 100, 200, or 500 features, including ranges between any two of the listed values. Example features suitable for some embodiments herein include posts formed from a polymer coating such as PTFE on the substrate (see, e.g. FIGS. 17A-B). In some embodiments, the thin-layer porous matrix is maintained in a substantially flat configuration and in close proximity to the surface via an attachment comprising at least one of threading, stitching, webbing, mesh, clips, or a fabric. Optionally, the substrate comprises a first mesh and a surface comprising a second mesh is positioned on a side of the thin-layer porous matrix opposite the substrate (i.e. the thin-layer porous matrix is sandwiched between two meshes) to maintain the layer in a position disposed over the substrate. Without being limited by any theory, it is contemplated that positioning a thin-layer porous matrix between a mesh substrate and a second mesh surface can facilitate washing and/or labeling of polynucleotides immobilized in the thin-layer porous matrix. In some embodiments, the attachment allows access to the thin-layer porous matrix. In some embodiments, the attachment allows an aqueous phase of the thin-layer porous matrix to be in fluid communication with an aqueous solution. The thin-layer porous matrix can be immersed in the aqueous solution. In some embodiments, the thin-layer porous matrix is not attached to the surface, or is reversibly attached to the surface. It is contemplated herein that when nucleic acids are covered from the thin-layer porous matrix, a detached matrix can facilitate the recovery.

### Substrates

A variety of substrates can be used in accordance with some embodiments herein.

In some embodiments, the substrate comprises a slide. Optionally, the slide is coated with a polymer, for example PTFE. Optionally, the polymer forms features of the substrate, for example two or more posts that can hold a thin-layer porous matrix in place on the substrate, for example, at least 2, 3, 4, 5, 6, 7, 8, 9, 10, 20, 30, 40, 50, 90, 100, 120, 150, 200, 250, 300, 400, 500, or 1000 posts, including ranges between any two of the listed values. FIGS. 17A and 17B are schematic diagrams showing PTFE features of PTFE coating rings on slides according to some embodiments herein. The PTFE features can comprise retaining posts for thin-porous layer matrices. FIG. 17A illustrates PTFE features 171 around an inside diameter of a PTFE ring 163 coated on a slide 162 for holding a thin-layer porous matrix in place during processing. FIG. 17B illustrates PTFE features 171 positioned uniformly over entire well inside PTFE ring 173 coated on a slide 172 for holding thin-layer porous matrix in place during processing.

In some embodiments, the substrate is rigid. In some embodiments, the substrate is flexible. In some embodiments, the substrate comprises a slide, container, or sheet.

In some embodiments, the substrate comprises a mesh, for example a nylon mesh, or a metallic mesh, or a polymer mesh such as a PTFE mesh. The mesh can comprise a plurality of openings. The plurality of openings can have a diameter of at least 0.1 nm, for example, 0.1 nm, 0.5, 1, 2, 5, 10, 20, 50, 100, 200, 500, 700, 900, 1000, 2000, 3000, 5000, 9000, or 10000 nm, including ranges between any two of the listed values, for example 0.1 nm to 10 mm, 0.1 nm to 1 mm, 0.1 nm to 500 nm, 0.1 nm to 100 nm, 0.1 nm to 10 nm, 1 nm to 10 mm, 1 nm to 1 mm, 1 nm to 500 nm, 1 nm to 100 nm, 1 nm to 10 nm 10 nm to 1 mm, 10 nm to 500 nm, 10 nm to 100 nm, 100 nm to 10 mm, or 100 nm to 1 mm. In some embodiments, the openings of the mesh have diameters of approximately the same size. In some embodiments, the openings of the mesh have diameters of different sizes.

### Polynucleotides

A variety of polynucleotides can be processed in accordance with embodiments herein. Genomic polynucleotides can include DNA, RNA, or a combination of DNA and RNA. As such, in some embodiments, the polynucleotide comprises DNA. In some embodiments, the polynucleotide comprises RNA. In some embodiments, the polynucleotide is double-stranded. In some embodiments, the polynucleotide is single-stranded. In some embodiments, the polynucleotide comprises a DNA-RNA hybrid. In some embodiments, the polynucleotide comprises at least about 100 kilobases (kb), for example at least about 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 15,000, 20,000, 30,000, or 40,000 kilobases, including ranges between any two of the listed values.

Genomic polynucleotides can comprise a variety of modifications, for example epigenetic modifications such as methylation. As such, in some embodiments, the polynucleotide comprises one or more modifications to a polynucleotide backbone, for example methylation. In some embodiments, one or more modifications have been removed from the polynucleotide.

### Biological Samples

A variety of biological samples comprising polynucleotides can be provided according to embodiments herein. In some embodiments, the sample comprises one or more whole cells. In some embodiments, the sample comprises one or more single-cell organisms. In some embodiments, the sample comprises one or more cells of a multicellular organism. In some embodiments, the sample comprises a tissue of a multicellular organism. In some embodiments, the sample comprises a combination of two or more cell types.

In some embodiments, the sample comprises at least one portion of at least one cell. In some embodiment, the sample comprises nucleic-acid-containing portions of the cell separated from other portions. For example, nuclei can be separated from other portions of the cell based on rate of sedimentation (e.g. through centrifugation).

In some embodiments, the sample comprises at least one of a cell suspension, a nuclei suspension, an organelle suspension, a cell homogenate, a tissue homogenate, a whole organism homogenate, and a biological fluid.

### Non-Polynucleotides, and Removal Thereof

As used herein “non-polynucleotides,” including variations of this root term, refers to any molecule, complex, or structure from a biological sample that is not a polynucleotide. Exemplary non-polynucleotides include biomolecules (other than polynucleotides) found in cells, including, but not limited to, polypeptides, amino acids, mononucleotides, carbohydrates, lipids, cofactors, inorganic molecules, organelles and components thereof, cellular debris, and the like. In some embodiments, the non-polynucleotides that are removed comprise at least one of a protein, a lipid, a carbohydrate, an organelle, and cellular debris.

Since the presence of non-polynucleotide molecules can interfere with manipulation, processing, and analysis of polynucleotides, in some embodiments, non-polynucleotide molecules are separated from the polynucleotide. In some embodiments, non-polynucleotide molecules are removed from the porous matrix while the polynucleotide remains in the porous matrix. In some embodiments, non-polynucleotides undergo modification, processing, and/or degradation prior to being removed from the porous matrix.

In some embodiments, the polynucleotide remains immobilized in the porous matrix while the non-polynucleotides are removed. In some embodiments, the polynucleotide remains within the matrix, but is not necessarily immobilized while the non-polynucleotides are removed.

Without being limited by any particular theory, non-polynucleotide molecules in the porous matrix can be modified or degraded to facilitate their exit from the porous matrix. As such, in some embodiments, removing the non-polynucleotide molecules comprises contacting the porous matrix with an elastase, a collagenase, a lipase, a carbohydratase, a pectinase, a pectolyase, an amylase, an RNase, a hyaluronidases, a chitinase, a gluculase, a lyticase, a zymolyase, a lysozyme, a labiase, an achromopeptidase, or a combination thereof. In some embodiments, removing the non-polynucleotide molecules comprises contacting the porous matrix with a proteinase. In some embodiments, removing non-polynucleotides comprises applying an electric field to remove at least some of the non-polynucleotides.

Purification of the polynucleotide can include washing non-polynucleotides, including modified or degraded non-polynucleotides out of the porous matrix. As such, in some embodiments, removing non-polynucleotide molecules comprises contacting the porous matrix with a detergent, a chaotrope, a buffer, a chelator, a water soluble organic solvent, a polymer (e.g. polyethylene glycol, polyvinypyrrolidone, polyvinyl alcohol, ethylene glycol), a salt, an acid, a base, a reducing agent, or a combination thereof. In some embodiments, removing the non-polynucleotide molecules comprises washing the porous matrix with a solution comprising, a buffer, a detergent, a chaotrope, a chelator, an alcohol, a salt, an acid, a base, a reducing agent, a polymer, or a combination thereof. In some embodiments, removing non-polynucleotide molecules comprises applying an electric field to remove at least some non-polynucleotide molecules.

In some embodiments, the polynucleotide is purified from the porous matrix without any labeling or characterization in the matrix. The polynucleotide can then be used for any of a variety of downstream applications known to the skilled artisan. It is contemplated that using methods as described herein, a particularly crude starting material (for example a whole cell extract) can be purified, and provide cleaner nucleic acid than purification using a standard plug, as the methods according to some embodiments herein provide enhance enzyme and chemical wash kinetics.

It can be useful to perform some processing of the sample in the porous matrix prior to removing non-polynucleotide molecules. For example, it can be useful to concentrate nucleic acid-containing components of the sample to a particular region prior to removing other components. In some embodiments, in-matrix nuclei enrichment is performed prior to removing non-polynucleotide molecules.

### Labeling

A variety of approaches and compositions for labeling polynucleotides can be used in accordance with some embodiments herein. In some embodiments, a polynucleotide undergoes site-specific labeling, for example to label one or more sequence motifs. In some embodiments, a polynucleotide undergoes non-site-specific labeling, for example to label a backbone. In some embodiments, the polynucleotide undergoes site-specific labeling and non-site specific labeling.

Labeling can be performed on a polynucleotide immobilized in a porous matrix as described herein, and/or can be performed after the polynucleotide is separated from the matrix. In some embodiments, the polynucleotide is labeled in the matrix. In some embodiments, the polynucleotide is separated from the matrix and then labeled. In some embodiments, the polynucleotide undergoes at least one labeling event in the matrix, and at least one labeling event after being separated from the matrix. For example, in some embodiments, the polynucleotide undergoes site-specific labeling within the matrix, and non-site-specific labeling after separation of the matrix.

In some embodiments, the polynucleotide is labeled by two or more labels, for example two, three, four, five, six, seven, eight, nine, or ten labels. In some embodiments, two or more of the labels are different. In some embodiments, two or more of the labels are the same. In some embodiments, the polynucleotide is labeled with at least one site-specific label, and at least one non-sequence-specific label that is different from the site-specific label. In some embodiments, the polynucleotide is labeled with two or more site-specific labels, and the non-sequence specific label is different from the site-specific labels.

In some embodiments, a first polynucleotide is labeled with a first label, and a second polynucleotide is labeled with a second label. In some embodiments, the first and second label are the same. In some embodiments, the first and second label are different. In some embodiments, a third polynucleotide is labeled with a third label. The third label can be the same as, or different from, either or both of the first and second label.

A variety of approaches for site-specific labeling can be used in accordance with embodiments herein. In some embodiments, the polynucleotide is contacted with a sequence-specific probe. In some embodiments, the polynucleotide is double-stranded, and site-specific labeling comprises nicking the polynucleotide at a first sequence motif, thus forming at least one nick so that the polynucleotide remains double-stranded adjacent to the nick or nicks, and labeling the nick or nicks with the first label. In some embodiments, at least one nucleotide is incorporated into the nick. The incorporate nucleotide can comprise a moiety that facilitates labeling, for example a reversible terminator, a reactive group, or the label itself.

Probes are suitably nucleic acids (single or multiple) that include a label, as described herein. In some embodiments, a probe is sequence-specific (e.g., AGGCT, or some other particular base sequence). In some embodiments, a probe is randomly generated. As described herein, a probe may be selected or constructed based on the user's desire to have the probe bind to a sequence of interest or, in one alternative, bind to a sequence that is upstream or downstream from a sequence or other region of interest on a particular DNA polymer (i.e., probes that bind so as to flank or bracket a region of interest). A probe may be as long as a flap (i.e., up to about 1000 bases). A probe is suitably in the range of from 1 to about 500 bases in length, or from about 1 to 100 bases or from about 3 to 50 bases, or even in the range of from about 5 to about 20 bases in length.

In some embodiments, a polynucleotide, for example an RNA or DNA, is labeled by hybridizing a probe to a single strand of the polynucleotide. The probe can be complementary to a strand of the RNA or DNA or a portion thereof. In some embodiments, the probe is complementary to a particular sequence motif. In some embodiments, a plurality of probes is provided so as to be complementary to a plurality of specific sequence motifs, for example at least 2, 3, 4, 5, 6, 7, 8, 9, 10, 20, 30, 40, 50, 60, 70, 80, 90, 100, 200, 300, 400, 500, 600, 700, 800, 900, 1000, 5,000, or 10,000 probes, including ranges between any two of the listed values. In some embodiments, the probe has a random sequence. In some embodiments, a probe with a plurality of random sequences is provided.

In some embodiments, a double-stranded DNA can be labeled by first melting hydrogen bonds between double stands of certain genomic regions to open a so-called D-loop, by increasing temperature or manipulation with organic solvent, and then hybridizing to at least one specific probes with equal or higher affinity to single stranded regions before annealing back to relative stable form. As such, in some embodiments, double-stranded DNA can be labeled by a probe as described herein without nicking either strand. In some embodiments, a plurality of D-loops can be opened on a single strand. As such, a plurality of probes can be annealed to a particular double-stranded DNA.

In some embodiments, labeling comprises transferring a label to the polynucleotide via a methyltransferase. In some embodiments, the methyltransferase specifically methylates a sequence motif. As such, labeling can comprise transferring a label to a sequence motif by the methyltransferase. Exemplary suitable DNA methyltransferases (MTase) include, but are not limited to, M.BseCI (methylates adenine at N6 within the 5′-ATCGAT-3′ sequence), M.Taq1 (methylates adenine at N6 within the 5′-TCGA-3′ sequence) and M.Hhal (methylates the first cytosine at C5 within the 5′-GCGC-3′ sequence). In some embodiments, two or more methyltransferases provide two or more labels, which can be the same or different.

In some embodiments, the site-specific labeling comprises transferring a reactive group to the first sequence motif; and coupling a label to the first reactive group.

In some embodiments, a double-stranded polynucleotide is labeled by first nicking the first strand of double-stranded polynucleotide. This nicking can be suitably effected at one or more sequence-specific locations, although the nicking can also be effected at one or more non-specific locations, including random or non-specific locations. Nicking can be suitably accomplished by exposing the double-stranded polynucleotide to a nicking endonuclease, or nickase. Nickases are suitably highly sequence-specific, meaning that they bind to a particular sequence of bases (motif) with a high degree of specificity. Nickases are available, e.g., from New England BioLabs (accessible on the world wide web at www.neb.com). Exemplary Nickases include, but are not limited to Nb.BbvCI; Nb.BsmI; Nb.BsrDI; Nb.BtsI; Nt.AlwI; Nt.BbvCI; Nt.BspQI; Nt.BstNBI; Nt.CviPII and combinations thereof. The nicking may also be accomplished by other enzymes that effect a break or cut in a polynucleotide strand. Such breaks or nicks can also be accomplished by exposure to electromagnetic radiation (e.g., UV light), one or more free radicals, and the like. Nicks may be effected by one or more of these techniques. In some embodiments, incorporation of replacement bases into the first strand (i.e., the nicked strand) of a double-stranded polynucleotide suitably comprises contacting the polynucleotide with a polymerase, one or more nucleotides, a ligase, or any combination thereof. In some embodiments, treating with a ligase following labeling of nicked polynucleotide can restore the integrity of the double-stranded polynucleotide and significantly increase the strength of the resulting strand.

In some embodiments, nickases that target the same sequence motif but nick at opposite strands are used to target specific DNA strands to minimize the formation of fragile sites. In some embodiments, nickases have been modified to only bind to one strand of a double-stranded DNA. In some embodiments, nickases are used to target a single strand from a first DNA molecule, and a single strand from a second DNA molecule. In some of these embodiments, a single strand from the first DNA is targeted by a first nickase, and the complementary strand from the second DNA molecule is targeted with a second nickase that recognizes the same sequence motif as the first nickase. In some embodiments, the orientation of extension is reversed for one of the strands. For example, in some embodiments, extension from the site of nicking occurs in one direction for a first DNA molecule, and in the opposite direction for a second DNA molecule. In some embodiments, extension from the site of nicking occurs in one direction for a top strand of a DNA molecule, and in the opposite direction for the bottom strand for the same DNA molecule.

In some embodiments, a double-stranded polynucleotide comprising a first polynucleotide strand and a second polynucleotide strand is processed to give rise to an unhybridized flap of the first polynucleotide strand and a corresponding region on the second polynucleotide strand, the unhybridized flap comprising from about 1 to about 1000 bases; extending the first polynucleotide strand along the corresponding region of the second polynucleotide strand; and labeling at least a portion of the unhybridized flap, a portion of the extended first polynucleotide strand. Labeling can be suitably accomplished by (a) binding at least one complementary probe to at least a portion of an unhybridized flap, the probe comprising one or more labels, (b) utilizing, as a replacement base that is part of the first polynucleotide strand extended along the corresponding region of the second polynucleotide strand, a nucleotide comprising one or more labels, or any combination of (a) and (b). In this way, the flap, the bases that fill-in the gap, or both may be labeled.

A variety of species can serve as labels, which can be used in methods provided herein. A label can include, for example, a fluorophore, a quantum dot, a dendrimer, a nanowire, a bead, a hapten, a streptavidin, an avidin, a neutravidin, a biotin, and a reactive group a peptide, a protein, a magnetic bead, a radiolabel, or a non-optical label. In some embodiments, the selected label is a fluorophore or a quantum dot.

In some embodiments, at least one label as described herein comprises a non-optical label. A variety of non-optical labels can be used in conjunction with embodiments herein. In some embodiments a non-optical label comprises an electronic label. Exemplary electronic labels include, but are not limited to molecule with a strong electric charge, for example ions such as a metal ions, charged amino acid side chain, or other cations or anions. An electronic label can be detected, for example, by conductivity (or resistivity) when the label is disposed in a detector. In some embodiments, a nanochannel comprises an electrode configured to determine the presence or absence of an electronic label by determining the conductivity or resistivity of a substance disposed in the channel. In some embodiments, the non-optical label comprises a metal, metal oxide (for example metal oxide), or silicon oxide moiety. In some embodiments, the non-optical label comprises a moiety (for example a nanoparticle) comprising a metal, metal oxide, or other oxide. The presence of a particular metal or oxide moiety can be detected, for example by nuclear magnetic resonance. In some embodiments, the label is configured to release a moiety, for example a proton or an anion, upon a certain condition (e.g. change of pH) and the presence or absence of released moiety is detected.

In some embodiments, site-specific labeling comprises contacting a sequence motif of the polynucleotide with a binding moiety that binds specifically to the sequence motif. A variety of binding moieties can be used in conjunction with embodiments herein. Exemplary binding moieties include, but are not limited to, a triple helix oligonucleotide, a peptide, a nucleic acid, a carbohydrate, a polyamide, a zinc finger DNA binding domain, a transcription activator like (TAL) effector DNA binding domain, a transcription factor DNA binding domain, a restriction enzyme DNA binding domain, an antibody, a combination of two or more of the listed binding moieties.

In some embodiments, a probe includes one or more of an organic fluorophore, quantum dot, dendrimer, nanowires, bead, Au beads, paramagnetic beads, magnetic bead, a radiolabel, polystyrene bead, polyethylene bead, peptide, protein, haptens, antibodies, antigens, streptavidin, avidin, neutravidin, biotin, nucleotide, oligonucleotide, sequence specific binding factors such as engineered restriction enzymes, methlytransferases, zinc finger binding proteins, and the like. In some embodiments, the probe includes a fluorophore-quencher pair. One configuration of the probe can include a fluorophore attached to the first end of the probe, and an appropriate quencher tethered to the second end of the probe. As such, when the probe is unhybridized, the quencher can prevent the fluorophore from fluorescing, while when the probe is hybridized to a target sequence, the probe is linearized, thus distancing the quencher from the fluorophore and permitting the fluorophore to fluoresce when excited by an appropriate wavelength of electromagnetic radiation. In some embodiments, a first probe includes a first fluorophore of a FRET pair, and a second probe includes a second fluorophore of a FRET pair. As such, hybridization of the first probe and the second probe to a single flap, or to a pair of flaps within a FRET radius of each other can permit energy transfer by FRET. In some embodiments, a first probe includes a first fluorophore of a FRET pair, and a label on a nucleotide incorporated to fill a corresponding gap can include second fluorophore of a FRET pair. As such, hybridization of the first probe to a flap, and the labeled nucleotide into the corresponding gap can permit energy transfer by FRET.

In some embodiments, the labeling comprises contacting the polynucleotide with a dye or stain. In some embodiments, the dye or stain is a non-sequence specific label, for example an intercalating agent. Exemplary non-sequence-specific labels that can be used in accordance with embodiments herein include YOYO, POPO, TOTO, SYBR Green I (Molecular Probes), PicoGreen (Molecular Probes), propidium iodide, ethidium bromide, and the like.

Separating Polynucleotides from Porous Matrices

After a polynucleotide has undergone desired processing in a porous matrix, it can be useful to separate the polynucleotide from the porous matrix, for example to analyze or characterize the polynucleotide.

In some embodiments, the polynucleotide is separated from the porous matrix after non-polynucleotides have been removed.

A variety of methods can be used to separate the polynucleotide can be separated from the porous matrix according to embodiments herein. In some embodiments, separating comprises at least one of melting the porous matrix, digesting the porous matrix, degrading the porous matrix, solubilizing the porous matrix, electroelution, spinning through a sieve, blotting onto a membrane, dialysis step, or a combination of two or more of the listed methods. In some embodiments, for example embodiments in which the porous matrix comprises agarose, the porous matrix is melted and contacted with agarase to separate the polynucleotides from the porous matrix. In some embodiments, after purifying and labeling DNA in matrix in a sequence specific manner, it is recovered by melting/digesting (e.g. for an agarose), dialyzed, and then mixed with flow buffer. In some embodiments, the flow buffer comprises a non-specific polynucleotide label, for example a DNA backbone staining dye (e.g. YOYO or POPO). The non-specific labeling can facilitate subsequent analysis, for example in a fluidic channel such as an Irys™ system (Bionano genomics).

In some embodiments, after the polynucleotide is separated from the porous matrix, the polynucleotide is analyzed and/or characterized. If the polynucleotide has been labeled with at least one site-specific label, a pattern of site-specific labeling characteristic of the polynucleotide can be detected. In some embodiments, patterns of two or more site-specific labels are detected. In some embodiments, patterns of site specific labeling are detected with reference to a non-specific label of the polynucleotide.

Approaches for detecting patterns of labeling in according with embodiments herein can comprise linearizing the polynucleotide. Means of linearizing a polynucleotide can include the use of shear force of liquid flow, capillary flow, convective flow, an electrical field, a dielectrical field, a thermal gradient, a magnetic field, combinations thereof (e.g., the use of physical confinement and an electrical field), or any other method known to one of skill in the art. In some embodiments, the polynucleotide is linearized and analyzed in a fluidic channel such a microchannel, or a nanochannel, for example the Irys™ system (Bionano Genomics). Examples of nanochannels and methods incorporating the use of nanochannels are provided in U.S. Publication Nos. 2011/0171634 and 2012/0237936, which are hereby incorporated by reference in their entireties.

In some embodiments, the fluidic channel comprises a microchannel. In some embodiments, the fluidic channel comprises a nanochannel. Suitable fluidic nanochannel segments have a characteristic cross-sectional dimension of less than about 1000 nm, less than about 500 nm, or less than about 200 nm, or less than about 100 nm, or even less than about 50 nm, about 10 nm, about 5 nm, about 2 nm, or even less than about than about 0.5 nm. A fluidic nanochannel segment suitably has a characteristic cross-sectional dimension of less than about twice the radius of gyration of the molecule. In some embodiments, the nanochannel has a characteristic cross-sectional dimension of at least about the persistence length of the molecule. Fluidic nanochannel segments suitable for the present invention have a length of at least about 100 nm, of at least about 500 nm, of at least about 1000 nm, of at least about 2 microns, of at least about 5 microns, of at least about 10 microns, of at least about 1 mm, or even of at least about 10 mm. Fluidic nanochannel segments are, in some embodiments, present at a density of at least 1 fluidic nanochannel segment per cubic centimeter.

Analysis can include comparing patterns of site-specific labeling on the polynucleotide to that of a reference polynucleotide. Such comparison can indicate the size of the polynucleotide, the presence or absence of mutations, genetic markers, and/or genomic rearrangement events such as deletions, duplication, inversions, and the like. In some embodiments, the pattern of a first label, second label, or a combination of the first and second label are compared to a pattern of labels on a reference DNA. In some embodiments, the pattern of the reference DNA is determined experimentally. In some embodiments, the pattern of the reference DNA is determined in silico.

In some embodiments, a plurality of patterns is assembled based on overlapping patterns of site-specific labeling, thereby constructing a polynucleotide map.

### Methods of Processing a Sample Comprising Polynucleotide

According to some embodiments, methods of processing a sample comprising a polynucleotide are provided. The method can comprise immobilizing the sample in a porous matrix. The method can comprise removing non-polynucleotide molecules from the porous while the polynucleotide remains in the matrix. In some embodiments, the non-polynucleotide molecules are removed from a thin-layer porous matrix. In some embodiments, the non-polynucleotide molecules are removed from the porous matrix, and the porous matrix is fragmented so as to forming a plurality of porous units. In some embodiments, non-polynucleotide molecules are removed from the matrix before fragmenting. In some embodiments, non-polynucleotide molecules are removed from the matrix before fragmenting. In some embodiments, non-polynucleotide molecules are removed from the matrix before fragmenting and after fragmenting. Without being limited by any theory, it has been observed that efficiency of removing non-polynucleotide molecules is generally higher when the removal is performed after fragmenting. In some embodiments, fragmented porous units are collected after the non-polynucleotide molecules are removed. By way of example, the porous units can be collected by centrifugation. In some embodiments, non-polynucleotide molecules are removed from the matrix before fragmenting and after fragmenting so as to optimize the kinetics of removing the non-polynucleotide molecules. In some embodiments, the polynucleotide is labeled. In some embodiments, the polynucleotide is separated from the matrix. In some embodiments, patterns of site-specific labeling are detected.

Without being limited by any particular theory, increasing the relative surface area of the porous matrix can improve the effectiveness and efficiency of removing non-polynucleotide molecules from the polynucleotide, and/or can improve the effectiveness and efficiency of labeling polynucleotides therein. Accordingly, in some embodiments, the porous matrix is in a configuration with relatively high surface area, for example as a thin layer, or as a plurality of porous units. In some embodiments, the sample is embedded in a precursor material, and the porous matrix is then formed into the desired shape or configuration. For example, the precursor material can be a liquid, and can be poured into a mold or chamber to form a porous matrix in a desired shape when the liquid forms a porous matrix. In some embodiments, the porous matrix is formed into a configuration with a high surface area (e.g. a thin layer or porous units), and the sample is then added to the matrix, for example by an electromagnetic field, or by diffusion.

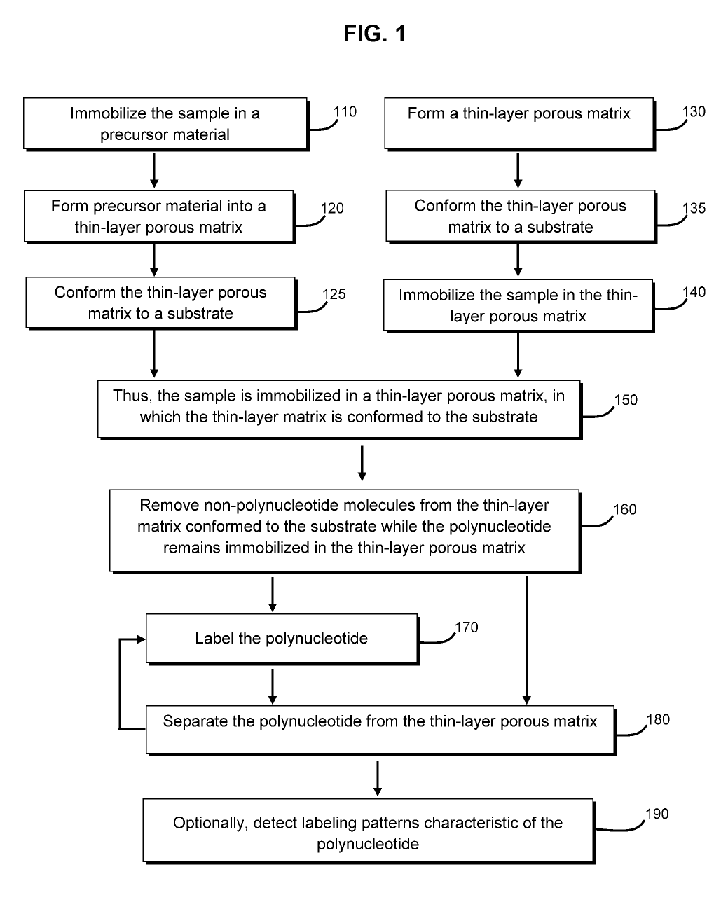

FIG. 1 illustrates a method of processing a sample comprising a polynucleotide according to some embodiments herein. In some embodiments, a sample is immobilized in a precursor material in 110. The precursor material can be formed into a thin-layer porous matrix in 120. The thin-layer porous matrix can be conformed to a substrate in 125. Optionally, the thin-layer porous matrix can be conformed to the substrate at the time the thin-layer porous matrix is formed. Optionally, the thin-layer porous matrix can be conformed to the substrate after thin-layer porous matrix is formed. In some embodiments, a thin-layer porous matrix is formed in 130. The thin-layer porous matrix can be conformed to a substrate in 135. Optionally, the thin-layer porous matrix can be conformed to the substrate at the time the thin-layer porous matrix is formed. Optionally, the thin-layer porous matrix can be conformed to the substrate after thin-layer porous matrix is formed. The sample can be immobilized in the thin-layer porous matrix in 140. The sample can thus be immobilized in a thin-layer porous matrix, in which the thin-layer matrix is conformed to a substrate in 150. The non-polynucleotide molecules can be removed from the thin-layer porous matrix disposed over the surface while the polynucleotide remains immobilized in the thin-layer porous matrix in 160. Optionally, the polynucleotide can be separated from the matrix in 170. The polynucleotide can be labeled in 180. Optionally, labeling patterns characteristic of the polynucleotide can be detected in 190. In some embodiments, the polynucleotide is labeled after it is separated from the matrix. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In some embodiments, the non-polynucleotide molecules are removed while the polynucleotide is immobilized in the thin-layer porous matrix disposed over a substrate as described herein. Optionally, the non-polynucleotide molecules are removed in the absence of an electric field applied to the thin-layer porous matrix (e.g. in the absence of electrophoresis). Optionally, the non-polynucleotide molecules are removed by washing, for example at least 1, 2, 3, 4, 5, 6, 7, 8, 9, 10, 15, 20, 30, 40, or 50 washes, including ranges between any two of the listed values. In some embodiments, the thin-layer porous matrix is in a substantially flat configuration while the non-polynucleotide molecules are removed.

In some embodiments, the non-polynucleotide molecules are removed while the polynucleotide is immobilized in the thin-layer porous matrix, while the substrate is embedded in the thin-layer porous matrix. Optionally, the thin-layer substrate comprises a mesh comprising a plurality of openings having a diameter of at least 0.1 nm, for example, 0.1 nm, 0.5, 1, 2, 5, 10, 20, 50, 100, 200, 500, 700, 900, 1000, 2000, 3000, 5000, 9000, or 10000 nm, including ranges between any two of the listed values, for example 0.1 nm to 10 mm, 0.1 nm to 1 mm, 0.1 nm to 500 nm, 0.1 nm to 100 nm, 0.1 nm to 10 nm, 1 nm to 10 mm, 1 nm to 1 mm, 1 nm to 500 nm, 1 nm to 100 nm, 1 nm to 10 nm 10 nm to 1 mm, 10 nm to 500 nm, 10 nm to 100 nm, 100 nm to 10 mm, or 100 nm to 1 mm. Without being limited by any theory, it is contemplated that the embedded mesh in accordance with some embodiments herein can provide the thin-layer porous matrix with rigidity to remain in an extended format to keep the thin-layer porous matrix exposed on multiple sides to a fluidic environment for better reaction kinetics. Without being limited by any theory, it is contemplated that the embedded mesh in accordance with some embodiments herein can facilitate labeling and/or washing of polynucleotide within thin-layer porous matrix. Optionally, the non-polynucleotide molecules are removed in the absence of any electric field applied to the thin-layer porous matrix (e.g. in the absence of electrophoresis). Optionally, the non-polynucleotide molecules are removed by washing, for example at least 1, 2, 3, 4, 5, 6, 7, 8, 9, 10, 15, 20, 30, 40, or 50 washes, including ranges between any two of the listed values.

In some embodiments, at least some labeling is performed while the polynucleotide is immobilized in the thin-layer porous matrix disposed over a substrate as described herein. In some embodiments, the thin-layer porous matrix is in a substantially flat configuration while the labeling is performed.

In some embodiments, the polynucleotide is separated from the porous matrix and then labeled. In some embodiments, the polynucleotide is labeled, and then separated from the porous matrix. In some embodiments, the polynucleotide is labeled with a first label in the porous matrix, removed from the porous matrix, and then labeled with a second label. In some embodiments, the first label is site-specific, and the second label is non-sequence specific. In some embodiments, the first label is non-sequence-specific, and the second label is site-specific.

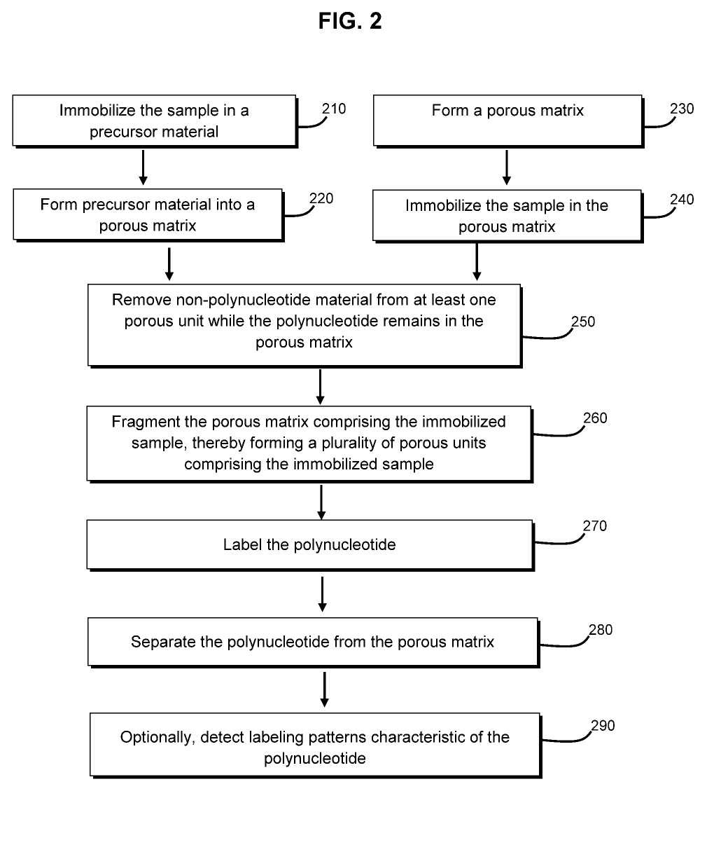

FIG. 2 illustrates a method of processing a sample comprising a polynucleotide according to some embodiments herein. In some embodiments, a sample is immobilized in a precursor material 210. The precursor material can be formed into a porous matrix 220. In some embodiments a porous matrix is formed 230. The sample can be immobilized in the porous matrix 240. In some embodiments, the non-polynucleotide material is removed from the porous matrix while the polynucleotide remains in the porous matrix 250. In some embodiments, the polynucleotide remains immobilized in the porous matrix while the non-polynucleotide material is removed. In some embodiments, the porous matrix comprising the immobilized sample, thereby forming a plurality of porous units comprising the immobilized sample 260. In some embodiments, the polynucleotide is labeled 270. Optionally, the polynucleotide can be separated from the matrix 280. Optionally, labeling patterns characteristic of the polynucleotide can be detected 290.

As used herein, “porous unit” and variations of this root term refers to a fragment of porous matrix having a volume of no more than about 1000 nanoliters, for example, no more than about 1000 nanoliters, 950, 900, 850, 800, 750, 700, 650, 600, 550, 500, 450, 400, 350, 300, 250, 200, 150, 100, 50, 40, 30, 20, 10, 5, or 1 nanoliters, including ranges between any two of the listed values.

A porous matrix can be fragmented into porous units formed by a variety of methods. In some embodiments, the porous matrix is ground or homogenized into porous units, for example via homogenization with a pestle. In some embodiments, the porous matrix is cut into porous units. In some embodiments, the porous matrix is mashed into porous units. In some embodiments, the porous matrix is partially digested with an enzyme, thus forming porous units.

### Fluidic Devices

In some embodiments, methods of processing a polynucleotide as described herein, are partially or entirely performed in a fluidic device. Fluidic devices can be useful for automatically or partially automatically performing the methods of processing polynucleotides described herein, for example controlling amounts and sequence of reactants that are added and/or removed, carrying out successive reactions and/or washes, modulating temperature, modulating pressure, modulating fluidic movement, and the like. Optionally, the fluidic device comprises a microfluidic device. Optionally, the fluidic device comprises a nanofluidic device. Example suitable fluidic devices include a mini-reactor as described in Mollova et al. (2009) “An automated sample preparation system with mini-reactor to isolate and process submegabase fragments of bacterial DNA.” Analytical Biochemistry 391(2):135-43, which is hereby incorporated by reference in its entirety. In some embodiments, the entire method (e.g. the method of FIG. 1 and/or FIG. 2) is performed in the fluidic device. In some embodiments, portions of the method are performed in the fluidic device, such as removal of non-polynucleotide molecules, labeling, and/or separation of nucleotides from the porous matrix (e.g. thin-layer porous matrix or porous units).

In some embodiments, a mixture comprising sample and porous matrix (e.g. thin-layer porous matrix or porous units) precursor is prepared outside of the fluidic device, and a porous matrix (e.g. thin-layer porous matrix or porous units) is formed in the fluidic device, and the polynucleotide is processed in the fluidic device. Optionally, a mixture comprising sample and porous matrix (e.g. thin-layer porous matrix or porous units) precursor is added to the fluidic device (for example by injection), and the thin-layer porous matrix or porous units is/are formed in the fluidic device so that immobilization of the sample in a thin-layer porous matrix or porous units is performed in the device. By way of example, the thin-layer porous matrix can be formed by application of a vacuum or gentle pressure to the fluidic device. The immobilized polynucleotide can optionally be automatically processed in the fluidic device. Optionally, removal of non-polynucleotide molecules, labeling of the polynucleotide, and/or removal of the polynucleotide from the thin-layer porous matrix can be automatically performed within the fluidic device. Optionally, removal of the non-polynucleotide molecules can be in the absence of any electric field applied to the thin-layer porous matrix (e.g. in the absence of electrophoresis). Optionally, the non-polynucleotide molecules are removed by washing, for example at least 1, 2, 3, 4, 5, 6, 7, 8, 9, 10, 15, 20, 30, 40, or 50 washes, including ranges between any two of the listed values.

In some embodiments, a mixture comprising sample and porous matrix (e.g. thin-layer porous matrix or porous units) precursor is prepared inside of the fluidic device, and a porous matrix (e.g. thin-layer porous matrix or porous units) is formed in the fluidic device, and the polynucleotide is processed in the fluidic device. Optionally, sample is added to the fluidic device (for example by injection), and combined with the porous matrix or the porous matrix precursor within the fluidic device, so that immobilization of the sample in a thin-layer porous matrix (or porous units) is performed in the device. By way of example, the thin-layer porous matrix can be formed by application of a vacuum or gentle pressure to the fluidic device. Optionally, removal of non-polynucleotide molecules, labeling of the polynucleotide, and/or removal of the polynucleotide from the porous matrix (e.g. thin layer porous matrix or porous units) can be automatically performed within the fluidic device.

In some embodiments, a porous matrix (e.g. a thin layer porous matrix or porous units) is formed outside of the fluidic device as described herein, and positioned inside the fluidic device. Optionally, sample is immobilized in the thin layer porous matrix or porous units before it is positioned inside the fluidic device. For example, sample can be immobilized in the thin layer porous matrix or porous units by contacting sample with a precursor of the thin-layer porous matrix. The sample immobilized in the thin layer porous matrix or porous units can be automatically processed in the fluidic device. Optionally, removal of non-polynucleotide molecules, labeling of the polynucleotide, and/or removal of the polynucleotide from the thin-layer porous matrix can be automatically performed within the fluidic device.

### Preparations

According to some embodiments herein, polynucleotide preparations are provided. The preparation can include a processed or partially processed polynucleotide immobilized in a porous matrix as described herein.

In some embodiments, the preparation comprises a thin-layer porous matrix disposed over a substrate and a polynucleotide immobilized in the porous matrix, in which the polynucleotide is substantially isolated from non-polynucleotide cellular components, and in which the polynucleotide has been labeled or enzymatically modified while in the matrix.

In some embodiments, the polynucleotide includes a first label associated with a first sequence motif. In some embodiments, the polynucleotide also includes a second label associated with a second sequence motif, in which the second motif is different from the first motif, and the second label is the same as or different from the first label. In some embodiments, the label of the polynucleotide includes at least ones labeled oligonucleotide incorporated into a nick in a double-stranded DNA or RNA. In some embodiments, the polynucleotide comprises at least one binding moiety as described herein.

### Methods of Processing a Sample

According to some embodiments, a method of processing a sample is provided. The method can comprise immobilizing the sample in a thin-layer porous matrix disposed over a substrate as described herein. The method can comprise processing the sample immobilized in the substrate-associated layer to remove undesired components while at least one desired component remains immobilized in the sample. The method can comprise separating at least one desired component from the porous matrix. The method can include characterizing the at least one desired component. In some embodiments, the sample is a biological sample. In some embodiments, the desired components include at least one biomolecule or complex thereof, for example, a polynucleotide, a polypeptide, a lipid, a carbohydrate, or an organelle. In some embodiments, the undesired components include any cellular component or products other than the desired component or components.

### Systems

According to some embodiments, stems for processing a sample containing at least one polynucleotide are provided. The system can include a porous matrix or precursor material configured to be formed into a thin-layer porous matrix comprising the sample. The system can include a substrate for forming the thin-layer porous matrix. The system can include a means for maintaining the thin-layer porous-matrix substantially disposed over the substrate. Exemplary mechanical means for maintaining the disposition of the thin-layer porous-matrix include mesh such as nylon mesh, clamps, brackets, gaskets, threading, a netting, a gel, an adhesive, a peg, a screw, a brace, a scaffold, and the like.

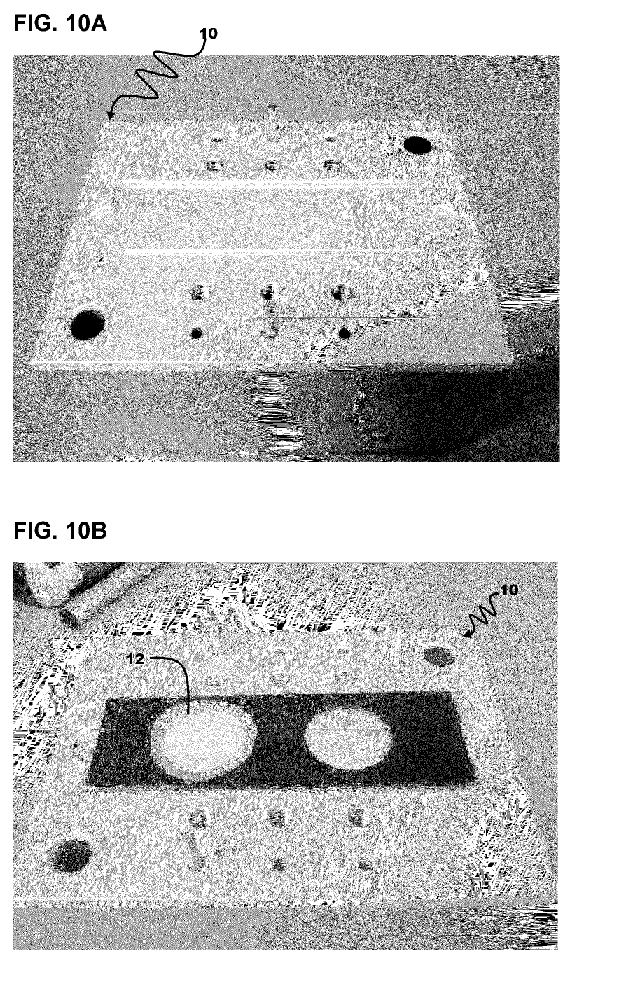

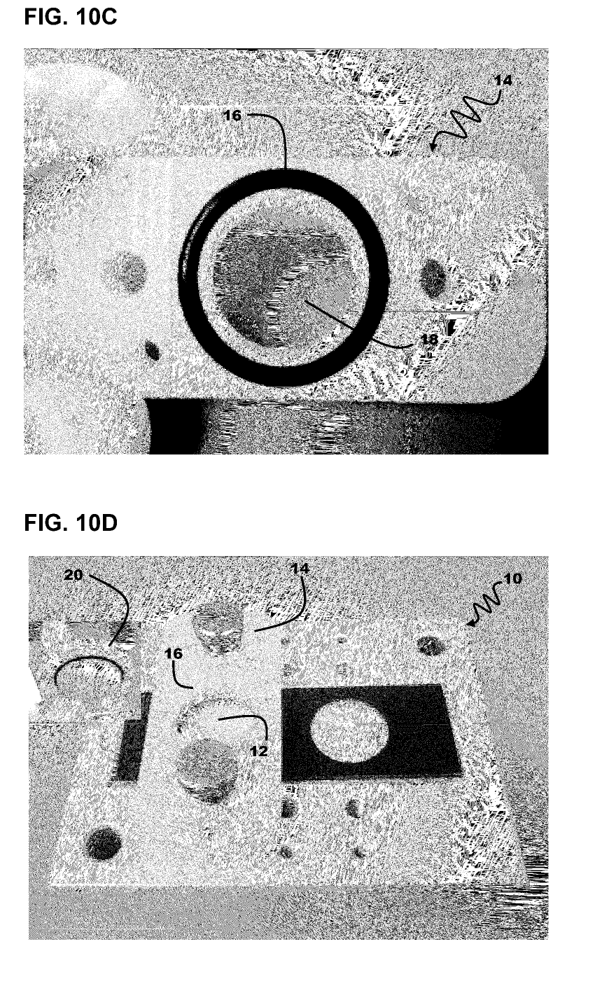

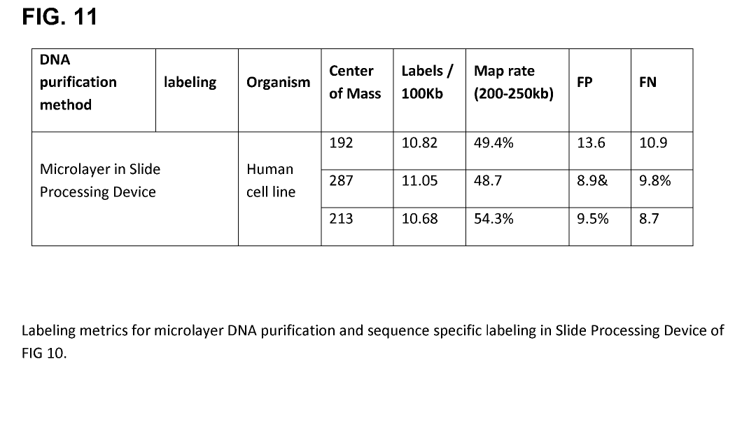

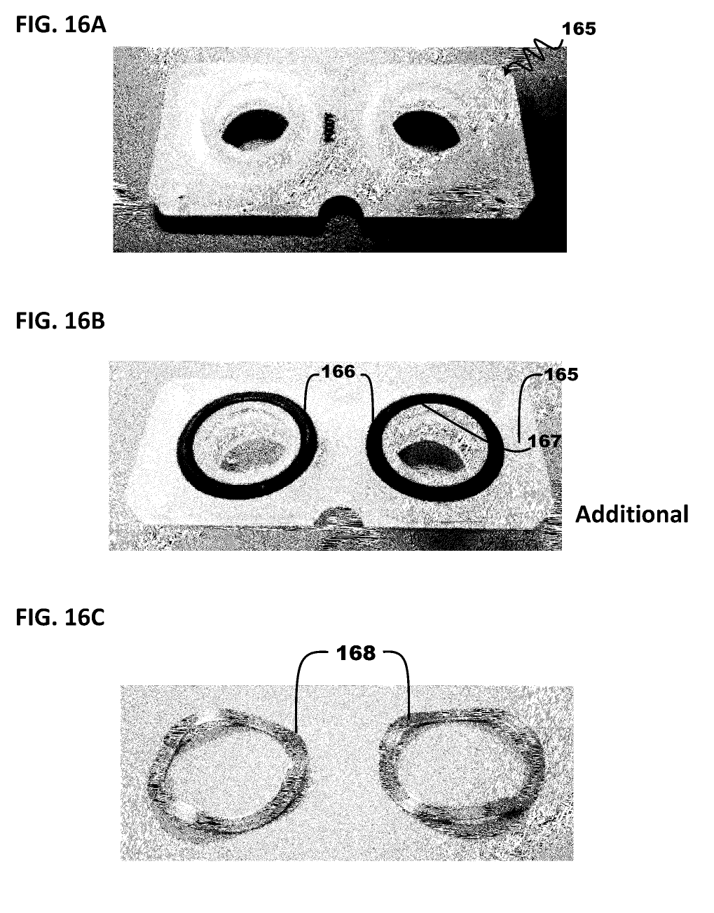

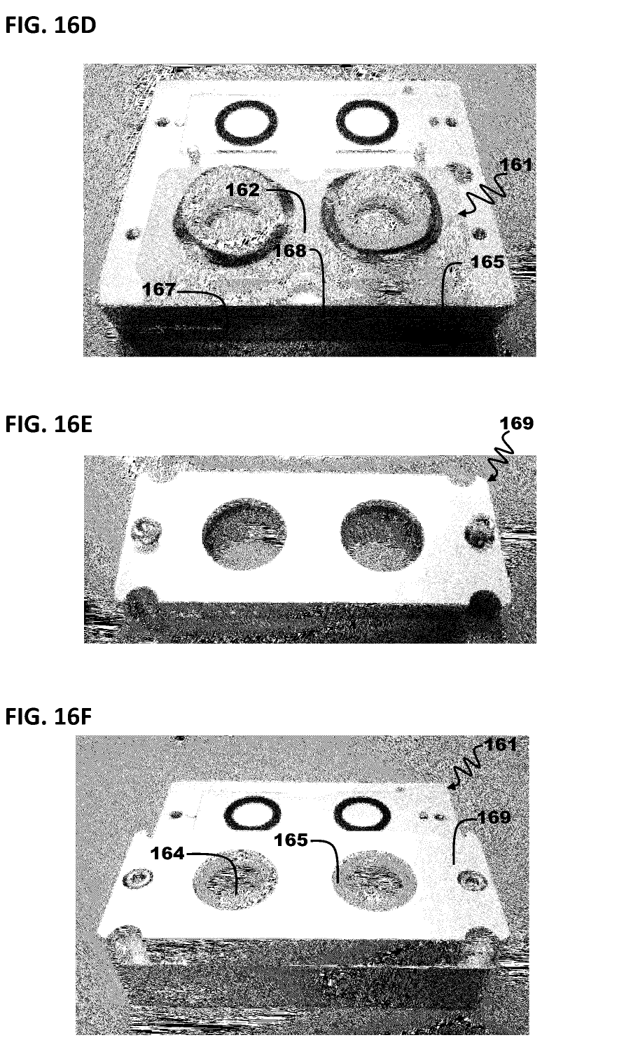

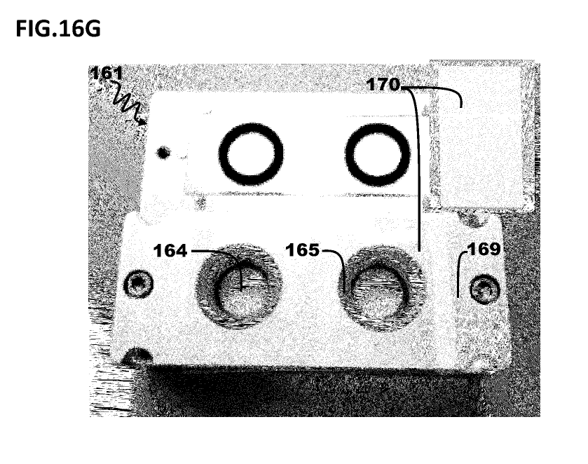

Some embodiments of a sample processing system are illustrated in FIGS. 10A-10D. The system can include a metallic base 10 for holding a slide that fits a heat block for temperature control. The system can include a substrate comprising a thin-layer porous matrix, for example a thin-layer porous matrix tethered with a nylon mesh 12 positioned over the base 10. The system can include a well-forming apparatus 14, including a reaction well 16 and o ring 18. The well forming unit 14 can be assembled over a substrate (for example a slide) comprising the thin-layer porous matrix tethered with a nylon mesh 12 and showing the reaction well 16. The system can include a lid 20 to close reaction well. Systems in accordance with some embodiments herein, and components of such systems are also illustrated in FIGS. 16A-16G, and FIGS. 17A-17B.

In some embodiments, the system comprises a mechanical means for forming a well around the thin-layer porous matrix. In some embodiments, the mechanical means comprises a well passing through the upper plate of the system, in which the gap between the upper plate and the lower plate of the system is tightened so that the thin-layer porous matrix is contained within the well. In some embodiments, a washer at the end of the well proximate to the thin-layer porous matrix minimizes leakage of matrix material, for example once the matrix material has been melted or digested.

In some embodiments, the system comprises a purification reagent for removing at least one non-polynucleotide as described herein.

In some embodiments, the system includes a first labeling reagent for labeling a sequence motif of the polynucleotide as described herein.

In some embodiments, the system includes a separation reagent for separating the labeled polynucleotide from the porous layer as described herein. As such, patterns of sequence motif labeling of the separated polynucleotide can be characterized.

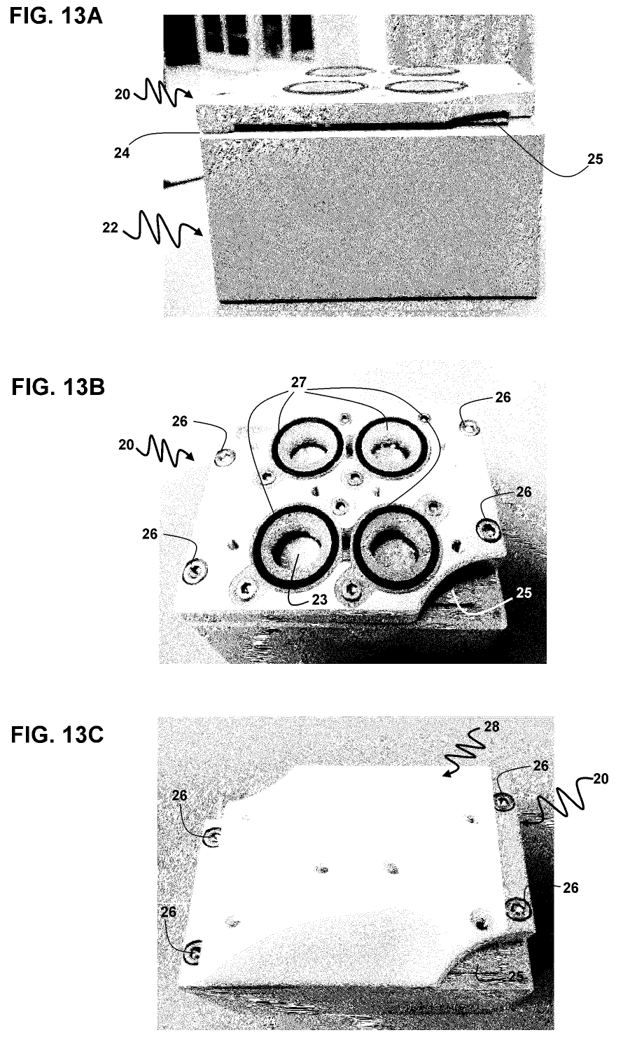

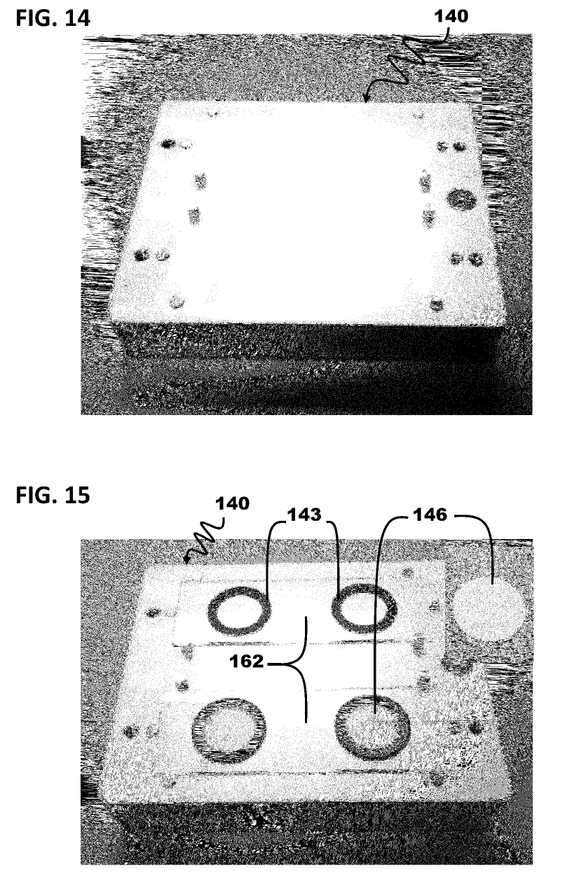

Some embodiments of a sample processing system are illustrated in FIGS. 13A-13C. It can be useful to maintain the thin-layer porous matrix at a particular temperature, for example to melt the thin-layer porous matrix. As such, the system can comprise an apparatus for heating the thin-layer porous matrix, while providing access to the thin-layer porous matrix. The system can comprise a metallic top plate 20. The system can comprise a metallic bottom plate 22. In some embodiments the metallic top plate 20 is positioned above the metallic bottom plate 22. In some embodiments the metallic top plate 20 is integrally formed with the metallic bottom plate 22, for example as a single piece of metallic material. In some embodiments the metallic top plate 20 is separate from the metallic bottom plate 22.

In some embodiments, the metallic top plate 20 is positioned above the metallic bottom plate 22 with a gap 24 therebetween. A thin-layer porous matrix disposed on a substrate can be disposed in the gap 24. In some embodiments, the gap 24 comprises a slit between the metallic top plate 20 and the metallic bottom plate 22. In some embodiments, the thin-layer porous matrix can be disposed upon a slide 25, which can be positioned in the gap 24. In some embodiments, the slide 25 comprises a polymer coating, for example a polytetrafluoroethylene coating. By positioning the thin-layer porous matrix in the gap 24, each side of the thin-layer porous matrix can be in contact with a metallic plate, thereby minimizing or eliminating any temperature gradient between various sides of the thin-layer porous matrix. As such, uniformity of heating, and uniformity of temperature can be improved over positioning the porous matrix on an open substrate. Moreover, positioning the porous matrix within the gap can minimize evaporation of liquids, as compared to an open-face arrangement. In some embodiment, the width of the gap 24 is adjustable. In some embodiments, the system comprises at least one tightener 26, for example a screw or bolt, which can be adjusted to increase or decrease the width of the gap 24 as desired. In some embodiments, the thickness of the gap 26 is adjusted to about the thickness of the slide.

In some embodiments, the metallic bottom plate 22 comprises a heating element, or is connected to a heating element via a conductive material. To improve uniformity of heating of the thin-layer porous matrix, at least a portion of the metallic top plate 20 can directly contact at least a portion of the metallic bottom plate 22. In some embodiments, the metallic top plate 20 comprises a heating element. In some embodiments, the metallic top plate 20 and metallic bottom plate 22 each comprises a heating element. In some embodiments, the metallic top plate 20 is detachable from the metallic bottom plate 22. In some embodiments, the metallic top plate 20 is attached to the metallic bottom plate 22 via a hinge. In some embodiments, the metallic top plate 20 is connected to the metallic bottom plate 22 via a track, configured so that the metallic top plate 20 can slide on and off of the metallic bottom plate 22.

So as to access a thin-layer porous matrix positioned in the gap 24, the metallic top plate 20 can comprise at least one well 27. The well 27 can comprise an opening that passes through the metallic top 20 plate, thereby providing access to the gap 23, and thus to provide access to a porous matrix within the gap 24. A plurality of wells 27 can increase throughput by allowing the processing of two or more porous matrices at a time. In some embodiments, a different thin-layer porous matrix can be disposed in each well 27. In some embodiments, the metallic top 20 comprises at least 2 wells, for example, 2, 3, 4, 5, 6, 7, 8, 9, 10, 11, 12, 13, 14, 15, 16, 17, 18, 19, 20, 25, 30, 35, 40, 45, or 50 wells, including ranges between any two of the listed values. In some embodiments, each well comprises an O-ring, for example as illustrated in FIG. 13B. In some embodiments, the wells have a substantially circular in shape. In some embodiments, the wells have a non-circular shape, for example oval, triangular, square, or any one of a number of polygons. In some embodiments, the opening of the wells 27 that is proximal to the gap 24 comprises a seal. In some embodiments, a slide 25 or other substrate can be inserted into the gap 24, and positioned under the wells so as to provide access to the porous matrix through the wells. Upon insertion of a slide 25 or other substrate into the gap 24, the width of the gap 24 can be adjusted so as to tighten the metallic bottom plate 22 and metallic top plate 20 around the slide 25 or other substrate. A width can be selected so as to minimize leakage from the wells 27 (seals on the well openings can also minimize leakage), and to minimize pressure on the slide 25 or other substrate so as to prevent cracking.

In some embodiments, the system comprises a cover 28. A cover 28 positioned over the wells 27 can retain heat within the wells 27, thereby improving uniform of heating. Moreover, a cover 28 can further prevent evaporation from the thin-layer porous matrix positioned at the bottom of a well 27, and can protect labeled molecules in the thin layer porous matrix from photobleaching.

In some embodiments, a plurality of systems comprise interchangeable parts, for example so that polytetrafluoroethylene slides and retaining plate bases, and seals over the slide are interchangeable between various systems (e.g. the system of FIGS. 10A-10D, the system of FIGS. 13A-13C, the system of FIGS. 16A-16G, and FIGS. 17A-17B).

### Methods and Kits for Forming Thin-Layer Porous Matrices

Thin-layer porous matrices in accordance with some embodiments herein can be formed by a variety of methods.

In some embodiments, a precursor of the thin-layer porous matrix is provided in a liquid state (e.g. at a temperature sufficient to melt the precursor, and typically greater than the ambient temperature). Sample can be contacted with the precursor. Optionally, the precursor and sample can be mixed. The precursor can be disposed over a surface and cooled, thus forming a thin-layer porous matrix. Optionally, the precursor is disposed directly over a substrate and cooled. Optionally, the precursor is disposed directly over a heated substrate and the substrate is subsequently cooled. By way of example, the substrate can comprise a mesh. Optionally, the substrate comprises one or more features such as posts that can hold the thin-layer porous matrix in place once it is disposed over the substrate. Optionally, the precursor is formed over a surface other than the substrate, cooled, and subsequently moved to the substrate. Optionally, the liquid precursor is spread across a mesh substrate so as to embed the mesh substrate in the thin-layer porous matrix.

In some embodiments, a precursor of the thin-layer porous matrix is provided, and centrifuge force is applied to the precursor to form a thin-layer porous matrix. Without being limited by any theory it is contemplated that centrifuge force can flatter an precursor to form a thin-layer porous matrix. Centrifuge force can be provided, for example, by a centrifuge. Optionally, the precursor can comprise sample prior to centrifugation. Optionally, sample can be added to the thin-layer porous matrix after centrifugation.

In some embodiments, a precursor of the thin-layer porous matrix is provided, and

vacuum or gas pressure is applied to the precursor to form a thin-layer porous matrix. For example, compressed gas can be applied to the precursor to flatten it into a thin-layer porous matrix. Example suitable gases include air, nitrogen, or an inert gas such as argon or helium.

In some embodiments, a porous matrix is provided in a configuration other than a thin layer and subsequently formed into a thin later, for example by compressing, cutting, shaving, grinding, or dissolving portions of the porous matrix, or by centrifuging the porous matrix. Optionally, the porous matrix comprises immobilized sample before it is formed into a thin-layer porous matrix. Optionally, sample is immobilized in the porous matrix after it is shaped into a thin-layer porous matrix.

In some embodiments, a well-forming apparatus (see, e.g. 14 in FIGS. 10A-D, or 165 in FIGS. 16A-G) is provided, and placed in contact with a substrate (see, e.g. 162 in FIGS. 16A-G). The well-forming apparatus can comprise a plurality of openings, for example at least 2, 3, 4, 5, 5, 7, 8, 9, 10, 15, 20, 25, 30, 35, 40, 45, 50, or 100 openings, including ranges between any two of the listed values. One or more openings of the well-forming apparatus can define surfaces perpendicular to the substrate. In some embodiments the well-forming apparatus forms a seal against the substrate. Optionally, the well-forming apparatus comprises a sealing member such as an o-ring that defines the opening(s) of the well-forming apparatus. Optionally, the well-forming apparatus is disposed on a base configured to control the temperature of the well-forming apparatus, for example a metal base configured to fit over a heating element (see, e.g. 161 in FIG. 16D). Liquid precursor of the thin-layer porous matrix can be positioned inside the openings of the well-forming apparatus so as to dispose liquid precursor over the substrate. Optionally, the liquid precursor comprises sample. Optionally, sample is contacted with the liquid precursor after it has been positioned inside the openings of the well-forming apparatus. The precursor of the thin-layer porous matrix can be cooled on the substrate.

FIGS. 16A-G is a series of photographs illustrating a sample processing device #2 according to some embodiments herein. FIG. 16A illustrates a well forming unit 165. FIG. 16B illustrates a well forming unit 165, including a reaction well 167 and o-ring 166. FIG. 16C illustrates a wave washer 168. FIG. 16D illustrates the well forming unit 165 positioned on slide and comprising the thin-layer porous matrix tethered with a nylon mesh 164 and showing the wave washer 168 positioned over each reaction well 167. FIG. 16E illustrates a metallic compression plate 169. FIG. 16F illustrates the well forming unit 165 assembled on slide comprising the thin-layer porous matrix tethered with a nylon mesh 164 and showing the compression plate 169 positioned over well forming unit 165. FIG. 16G illustrates the positioning of an adhesive sealing film 170 to create top air seal.

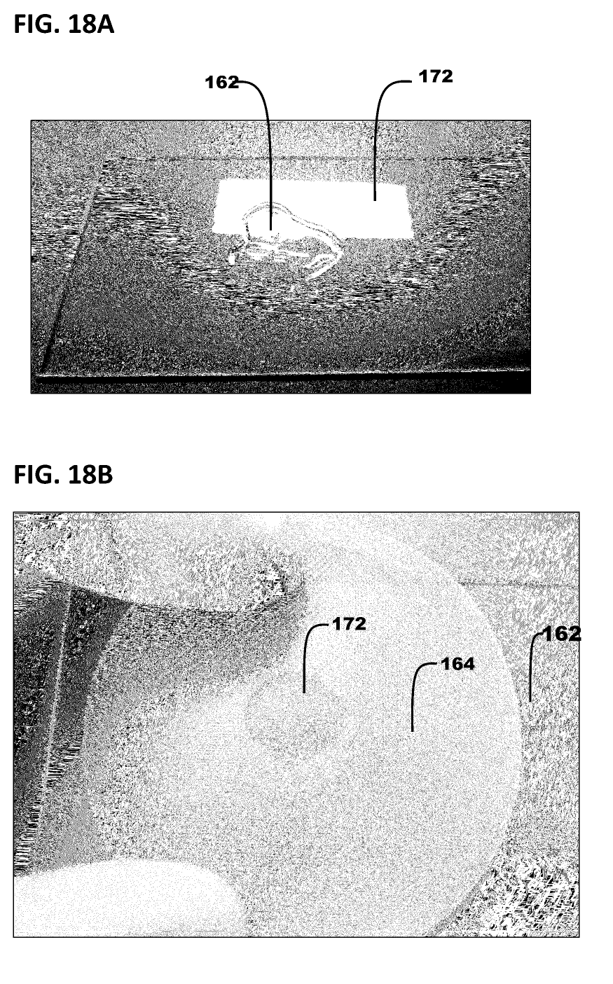

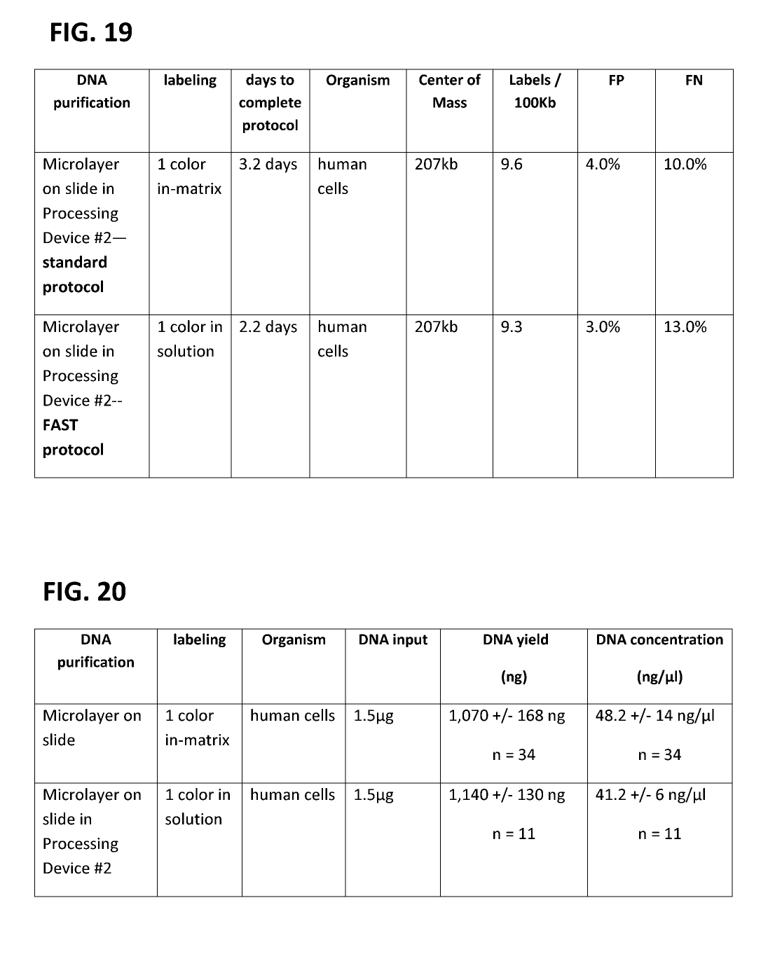

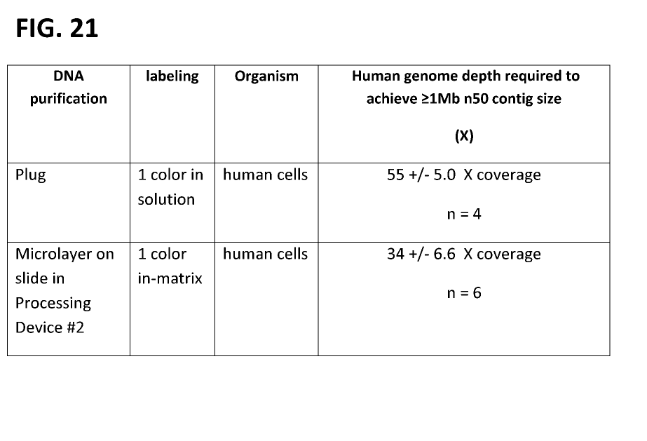

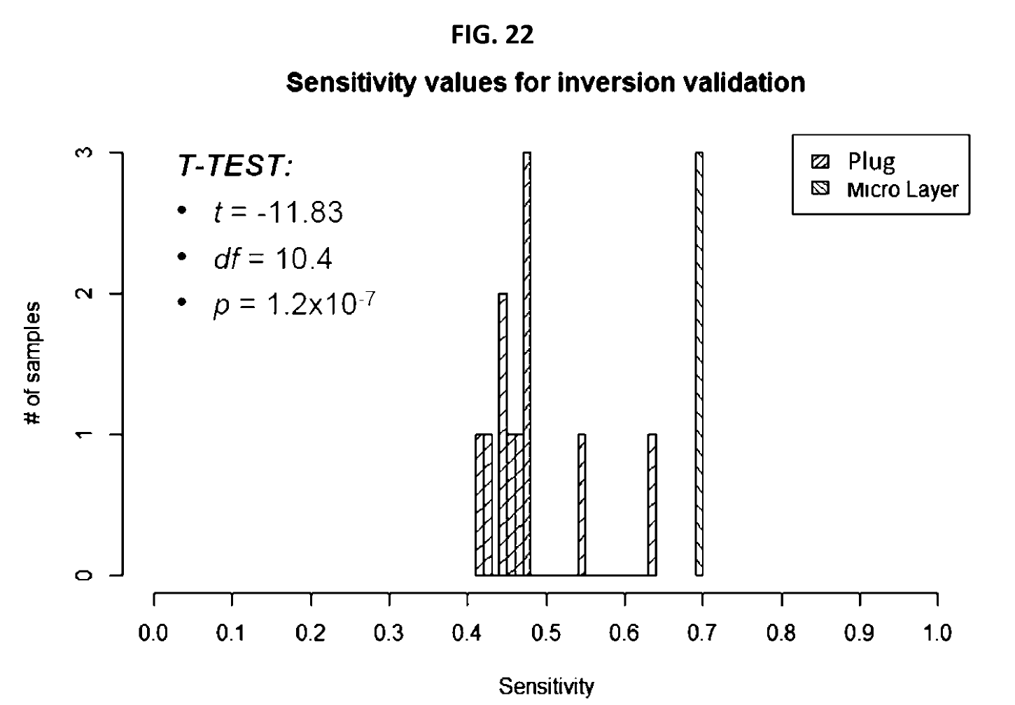

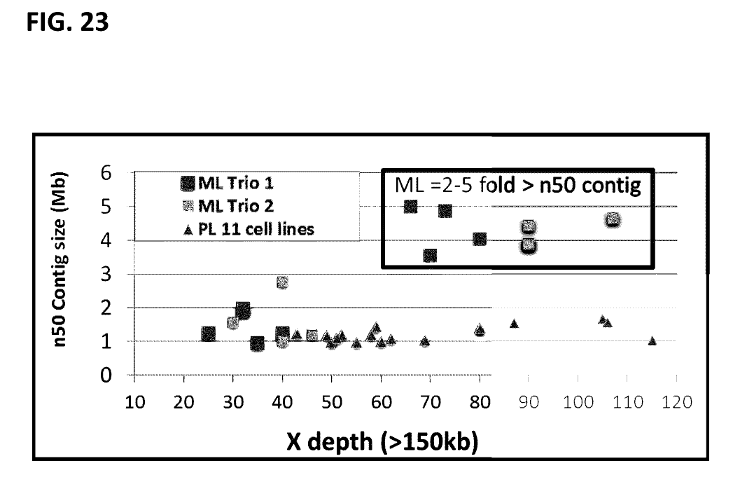

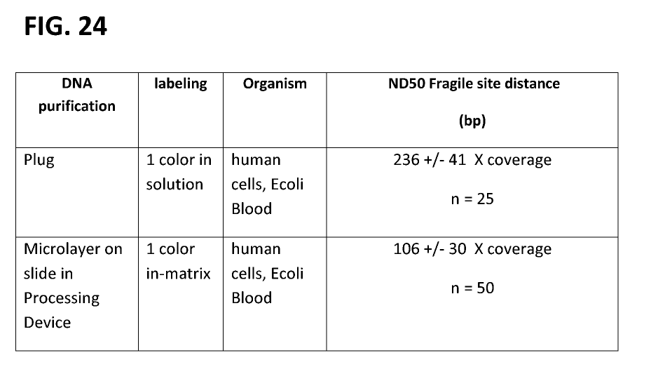

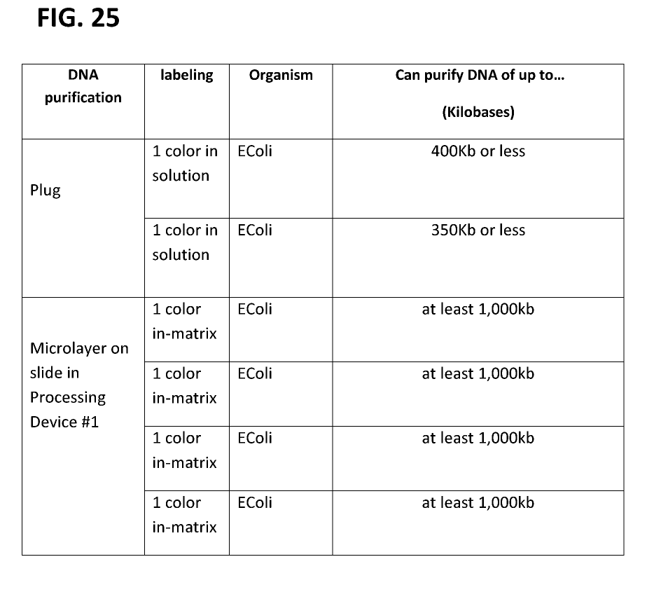

FIGS. 18A and 18B are photographs illustrating thin-layer porous matrices formed in accordance with some embodiments herein. FIG. 18A illustrates a thin-layer porous matrix on a slide. FIG. 18A illustrates the formation of a thin-layer porous matrix 172 on a slide 162 after compressed air was applied to precursor material. FIG. 18B illustrates a thin-layer porous matrix on a porous mesh substrate. FIG. 18B illustrates the formation of a thin-layer porous matrix 172 on mesh 164 after precursor material was compressed between two slides.

In some embodiments, a kit for forming a thin-layer porous matrix is provided. The kit can comprise a substrate as described herein. The kit can comprise a well-forming apparatus, in which the well-forming apparatus comprises one or more openings configured to define one or more surfaces perpendicular or substantially perpendicular to the substrate when placed against the substrate. Optionally, the well-forming apparatus comprises a sealing member such as an o-ring configured to form a seal against the substrate. Optionally, the kit further comprises a thin-layer porous matrix precursor. Optionally, the kit further comprises a compression plate. The compression plate can be configured to immobilize the well-forming apparatus against the substrate. The compression plate can further be configured to immobilize the well-forming apparatus and/or substrate directly or indirectly against a heating member. Optionally, the kit further comprises a heating member configured to heat the substrate and the well-forming apparatus. Optionally, the kit further comprises a mesh, for example a nylon mesh. Optionally, the kit further comprises a fluidic device, for example a microfluidic device or nanofluidic device. The fluidic device can be used for automatically processing polynucleotides in accordance with some embodiments herein. Optionally, the kit can be used for performing one or more of the methods of forming a thin-layer porous matrix as described herein. Optionally, the kit further comprises packaging and/or instructions for forming a thin-layer porous matrix. Example components of kits in accordance with some embodiments herein are illustrated in FIGS. 13A-C, 14, 15, 16A-G,17, and 18.

### ADDITIONAL EMBODIMENTS

According to some embodiments herein, method of processing a sample comprising a polynucleotide is provided. The method can comprise immobilizing the sample in a thin-layer porous matrix, wherein the thin-layer matrix is disposed over a substrate. The method can comprise removing non-polynucleotide molecules from the thin-layer matrix disposed over the substrate while the polynucleotide remains immobilized in the matrix. The method can comprise at least one of labeling the polynucleotide with a first label, or separating the polynucleotide from the thin-layer porous matrix. In some embodiments, the polynucleotide is labeled with a first label. In some embodiments, the polynucleotide is separated from the thin-layer porous matrix. In some embodiments, the polynucleotide is labeled with a first label and separated from the thin-layer porous matrix. In some embodiments, the polynucleotide is labeled with a first label and subsequently separated from the thin-layer porous matrix. In some embodiments, the polynucleotide is separated from the thin-layer porous matrix, and subsequently labeled with a first label. In some embodiments, the polynucleotide is labeled with the first label after removing non-polynucleotide molecules and before separating the polynucleotide from the matrix. In some embodiments, the polynucleotide is labeled with the first label, and the label is detected while the polynucleotide is still in the matrix. In some embodiments, immobilizing the sample in a thin-layer porous matrix comprises contacting the sample with a precursor material, and subsequently forming the precursor material into a thin layer, thereby immobilizing the sample in a thin-layer porous matrix. In some embodiments, the thin-layer porous matrix remains disposed substantially flattened over the substrate. In some embodiments, the thin-layer matrix has a thickness of about 1 to 999 micrometers. In some embodiments, the thin-layer matrix has a thickness of about 80 to 200 micrometers. In some embodiments, the thin-layer porous matrix is attached to the substrate. In some embodiments, the thin-layer porous matrix is detached from the substrate, but remains in close proximity to the substrate such that the layer remains substantially flat throughout the processing. In some embodiments, the thin-layer porous matrix is maintained in close proximity to the substrate via at least one of a tether, scaffold, electromagnetic interaction, friction, or pressure. In some embodiments, the thin-layer porous matrix is maintained in close proximity to the substrate via a tether. In some embodiments, the tether comprises a porous material configured to maintain the thin-layer porous in close proximity to the substrate while allowing access to the sample immobilized in the thin layer. In some embodiments, the substrate is rigid. In some embodiments, the substrate is flexible. In some embodiments, the substrate is that of a slide, a container or a sheet. In some embodiments, immobilizing the sample in a thin-layer porous matrix comprises forming the thin-layer porous matrix such that the substrate defines at least one side of the thin-layer porous matrix. In some embodiments, the thin-layer porous matrix is formed between the substrate and another entity, thereby defining at least one of a thickness, diameter, or volume of the thin-layer porous matrix.

In some embodiments, a method of processing a sample comprising a polynucleotide is provided. The method can comprise immobilizing the sample in a porous matrix. The method can comprise fragmenting the porous matrix. The method can comprise removing non-polynucleotide molecules from the porous matrix while the polynucleotide remains in the porous matrix. The method can comprise separating the polynucleotide from the porous matrix. In some embodiments, non-polynucleotide molecules are removed from the porous matrix after fragmenting the porous matrix. In some embodiments, non-polynucleotide molecules are removed from the porous matrix prior to fragmenting the porous matrix. In some embodiments, the method further comprises removing traces of non-polynucleotide molecules from the porous matrix after fragmenting the matrix, wherein polynucleotide molecules remain in the porous matrix while the traces of non-polynucleotide molecules are removed. In some embodiments, the method further comprises labeling the polynucleotide with a first label after removing non-polynucleotide molecules from the porous matrix and before separating the polynucleotide from the matrix.

In some embodiments, for any of the methods described herein the polynucleotide comprises at least about 200 kilobases, for example, at least about 200 kb, 250 kb, 300 kb, 350 kb, 400 kb, 450 kb, 500 kb, 550 kb, 600 kb, 650 kb, 700 kb, 750 km 850 kb, 950 kb or 1000 kb, including ranges between any two of the listed values. In some embodiments, for any of the methods described herein the polynucleotide comprises at least about 1 megabase. In some embodiments, for any of the methods described herein the sample comprises at least one of a cell suspension, a nuclei suspension, an organelle suspension, a cell homogenate, a tissue homogenate, a whole organism homogenate, and a biological fluid. In some embodiments, for any of the methods described herein, the sample comprises a whole cell. In some embodiments, for any of the methods described herein, the polynucleotide comprises single-stranded DNA, single-stranded RNA, double-stranded DNA, or double-stranded RNA. In some embodiments, the porous matrix comprises a synthetic polymer, a naturally occurring polymer, or a combination thereof.

In some embodiments, for any of the methods described herein the porous matrix comprises a polysaccharide-based matrix. In some embodiments, for any of the methods described herein, the porous matrix comprises an agarose matrix, a polyacrylamide matrix, a gelatin matrix, a collagen matrix, a fibrin matrix, a chitosan matrix, an alginate matrix, a hyaluronic acid matrix, or any combination thereof. In some embodiments, for any of the methods described herein the porous matrix comprises an agarose matrix. In some embodiments, for any of the methods described herein the porous matrix comprises a silane group, a positively charged group, a negatively charged group, a zwitterionic group, a polar group, a hydrophilic group, a hydrophobic group, or any combination thereof. In some embodiments, for any of the methods described herein, the porous matrix comprises an aqueous environment. In some embodiments, for any of the methods described herein, the porous matrix is disposed in an aqueous solution. In some embodiments, for any of the methods described herein, non-polynucleotide molecules comprise at least one of a protein, a lipid, a carbohydrate, an organelle, and cellular debris. In some embodiments, for any of the methods described herein, removing non-polynucleotide molecules comprises contacting the porous matrix with a proteinase, an elastase, a collagenase, a lipase, a carbohydratase, a pectinase, a pectolyase, an amylase, an RNase, a hyaluronidases, a chitinase, a gluculase, a lyticase, a zymolyase, a lysozyme, a labiase, an achromopeptidase, or a combination thereof. In some embodiments, for any of the methods described herein, removing non-polynucleotide molecules comprises contacting the porous matrix with a proteinase. In some embodiments, for any of the methods described herein, removing non-polynucleotide molecules comprises contacting the porous matrix with a detergent, a chaotrope, a buffer, a chelator, an organic solvent, a polymer (e.g. polyethylene glycol, polyvinypyrrolidone, polyvinyl alcohol, ethylene glycol), a salt, an acid, a base, a reducing agent, or a combination thereof. In some embodiments, for any of the methods described herein removing non-polynucleotide molecules comprises washing the porous matrix with a solution comprising, a buffer, a detergent, a chaotrope, a chelator, an organic solvent, an alcohol, a salt, an acid, a base, a reducing agent, a polymer, or a combination thereof. In some embodiments, the organic solvent is miscible in an aqueous based solution. In some embodiments, for any of the methods described herein, removing non-polynucleotide molecules comprises applying an electric field to remove at least some non-polynucleotide molecules. In some embodiments, any of the methods described herein further comprises in-matrix nuclei enrichment prior to removing non-polynucleotide molecules.

In some embodiments, for any of the methods described herein, the labeling comprises non-site-specific labeling, for example with a YOYO or POPO dye.

In some embodiments, for any of the methods described herein, the labeling comprises site-specific labeling. In some embodiments, for any of the methods described herein labeling comprises contacting the polynucleotide with a dye or stain. In some embodiments, for any of the methods described herein, the labeling comprises non-optical labeling. In some embodiments, for any of the methods described herein, the polynucleotide is double-stranded, and site-specific labeling comprises nicking the polynucleotide at a first sequence motif, so as to form at least one nick, in which the DNA remains double-stranded adjacent to the at least one nick, and labeling the at least one nick with the first label. In some embodiments, the polynucleotide is immobilized in the matrix when nicked. In some embodiments, the site-specific labeling comprises incorporating at least one nucleotide into the at least one nick. In some embodiments, the at least one nucleotide comprises a reversible terminator. In some embodiments, the at least one nucleotide comprises the first label. In some embodiments, the site-specific labeling further comprises nicking the polynucleotide at a second sequence motif, thereby forming at least one second nick, wherein the DNA remains double-stranded adjacent to the at least one second nick, and labeling the at least one second nick with a second label, wherein the first label and the second label are the same or different. In some embodiments, for any of the methods described herein, the labeling comprises transferring the label to the polynucleotide by a first methyltransferase. In some embodiments, site-specific labeling comprises transferring the first label to a first sequence motif by a first methyltransferase. In some embodiments, site-specific labeling comprises transferring a first reactive group to the first sequence motif, and coupling the first label to the first reactive group. In some embodiments, site-specific labeling further comprises transferring a second label to a second sequence motif by a second methyltransferase, wherein the second sequence motif is different from the first sequence motif, and wherein the second label is the same or different from the first label. In some embodiments, site-specific labeling comprises contacting a first sequence motif of the polynucleotide immobilized in the matrix with a first binding moiety that binds specifically to the first sequence motif. In some embodiments, the first binding moiety comprises one of a triple helix oligonucleotide, a peptide, a nucleic acid, a polyamide, a zinc finger DNA binding domain, a transcription activator like (TAL) effector DNA binding domain, a transcription factor DNA binding domain, a restriction enzyme DNA binding domain, an antibody, or any combination thereof. In some embodiments, at least one of the first label or the second label is selected from the group consisting of a fluorophore, a quantum dot, or a non-optical label. In some embodiments, for any of the methods described herein, the method further comprising labeling the polynucleotide with a non-sequence-specific label, wherein the non-sequence specific label is different from the first and second labels.

In some embodiments, for any of the methods described herein, separating comprises at least one of melting the porous matrix, digesting the porous matrix, degrading the porous matrix, solubilizing the porous matrix, electroelution, spinning through a sieve, blotting onto a membrane, dialysis step, or a combination thereof. In some embodiments separating comprises adding a solvent to a mixture comprising the polynucleotide and at least one component of the matrix.

In some embodiments, any of the methods described herein, further comprises detecting a pattern of site-specific labeling characteristic of the polynucleotide. In some embodiments, detecting comprises linearizing the polynucleotide in a fluidic channel. In some embodiments, detecting comprises comparing a pattern of the first label, second label or any combination thereof to a pattern of labels on a reference DNA. In some embodiments, detecting comprises assembling a plurality of patterns based on overlapping patterns of site-specific labeling, thereby constructing a polynucleotide map.

According to some embodiments herein, a polynucleotide preparation is provided. The preparation can comprise a thin-layer porous matrix disposed over a substrate. The preparation can comprise a polynucleotide immobilized in the porous matrix, in which the polynucleotide is substantially isolated from non-polynucleotide cellular components, and wherein the polynucleotide has been site-specifically labeled or enzymatically modified while in the matrix. In some embodiments, the polynucleotide was separated from cellular components while in the matrix. In some embodiments, the polynucleotide was labeled prior to separation from cellular components. In some embodiments, the polynucleotide was labeled after separation from cellular components. In some embodiments, the polynucleotide comprises at least about 200 kilobases, for example, at least about 200 kb, 250 kb, 300 kb, 350 kb, 400 kb, 450 kb, 500 kb, 550 kb, 600 kb, 650 kb, 700 kb, 750 km 850 kb, 950 kb or 1000 kb, including ranges between any two of the listed values. In some embodiments, the polynucleotide comprises at least about 1 megabase. In some embodiments, the polynucleotide comprises single-stranded DNA, single-stranded RNA, double-stranded DNA, or double-stranded RNA. In some embodiments, the porous matrix comprises a synthetic polymer, a naturally occurring polymer, or a combination thereof. In some embodiments, the porous matrix comprises a polyacrylamide matrix, a gelatin matrix, a collagen matrix, a fibrin matrix, a chitosan matrix, an alginate matrix, a hyaluronic acid matrix, or any combination thereof. In some embodiments, the thin-layer porous matrix comprises an agarose matrix. In some embodiments, the thin-layer porous matrix comprises a polysaccharide-based matrix. In some embodiments, the porous matrix comprises a silane, a positively charged group, a negatively charged group, a zwitterionic group, a polar group, a hydrophilic group, a hydrophobic group, or any combination thereof. In some embodiments, the thin-layer porous matrix is disposed over the substrate in an extended configuration. In some embodiments, the thin-layer matrix has a thickness of about 1 to 999 micrometers. In some embodiments, the thin-layer porous matrix has a thickness of about 80 to 200 micrometers. In some embodiments, the thin-layer porous matrix is immobilized on the substrate. In some embodiments, the thin-layer porous matrix is detached from the substrate, but remains in close proximity to the substrate such that the layer remains substantially extended throughout the processing. In some embodiments, the substrate is rigid. In some embodiments, the substrate is flexible. In some embodiments, the substrate is that of a slide, a container or a sheet. In some embodiments, the thin-layer porous matrix is substantially free of non-polynucleotide cellular components. In some embodiments, the non-polynucleotide cellular components comprise at least one of proteins, lipids, carbohydrates, organelles, and cellular debris. In some embodiments, the site-specific labeling or enzymatic modification comprises labeling with at least a first label associated with a first sequence motif. In some embodiments, the site-specific labeling or enzymatic modification further comprises labeling with a second label associated with a second sequence motif, wherein the second label is the same as or different from the first label. In some embodiments, the site-specific labeling comprises labeling with at least a labeled oligonucleotide incorporated into a nick in a double-stranded DNA or RNA. In some embodiments, the preparation further comprises at least one binding moiety bound to the first motif, in which the binding moiety comprises at least one of a triple helix oligo, a peptide nucleic acid, a polyamide, a zinc finger DNA binding domain, a transcription activator like (TAL) effector DNA binding domain, a transcription factor DNA binding domain, a restriction enzyme DNA binding domain, an antibody, or a combination of any of these. In some embodiments, the site specific labeling comprise labeling with a label selected from the group consisting of a fluorophore, a quantum dot, and a non-optical label.

According to some embodiments herein, a system for processing a sample containing at least one polynucleotide is provided. The system can comprise a porous matrix configured to be formed into a thin-layer porous matrix comprising the sample. The system can comprise a substrate for forming the thin-layer porous matrix. The system can comprise a means for maintaining the thin-layer porous-matrix substantially disposed over the substrate. In some embodiments, the system further comprises a mechanical means for forming a well around the thin-layer porous-matrix substantially disposed over the substrate. In some embodiments, the system further comprises a means for maintaining the thin-layer porous matrix at a desired temperature. In some embodiments, the system further comprises a purification reagent for removing a sample component other than the at least one polynucleotide, a first labeling reagent for labeling a sequence motif of the at least one polynucleotide with a first label; and a separation reagent for separating the labeled polynucleotide from the thin-layer porous matrix, wherein patterns of sequence motif labeling of the separated polynucleotide can be characterized.

### Example 1

### Thin Layer-Based DNA Purification on a Slide Followed by in-Matrix One or Two Color Labeling

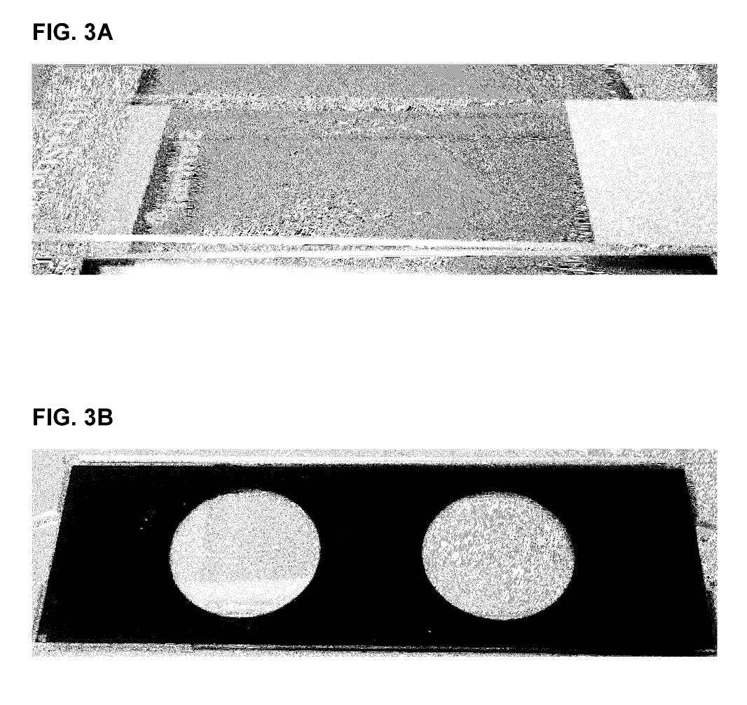

E. coli cells were mixed with an agarose solution and spread on a glass slide by sandwiching with another slide in the presence of 80 μm spacers. Upon solidification of the agarose-E. coli matrix at 4° C., the top sandwiching slide was removed leaving a porous microlayer attached to the bottom slide (FIG. 3). The attached microlayer was treated with lysozyme and proteinase K followed by several washes to remove contaminants leaving clean DNA behind. DNA retained in the microlayer was nicked with Nt.BspQI, washed, and labeled by nick translation with taq polymerase in the presence of green fluorescent dye coupled to dUTP. Following another wash step, the labeled nicks were repaired by treating with PreCR (New England BioLabs). For two color labeling the nicked, labeled, and repaired DNA retained in the microlayer was subjected to another round of nicking with a different nickase (Nb.BbvCI), labeling with a red fluorescent dye coupled to dUTP, and repairing with PreCR with washes in between. The microlayer-containing DNA was liquefied by melting the agarose and treating it with agarase to liberate the DNA which was stained with YOYO and processed on the Irys™ system (BioNano Genomics). Briefly, DNA was linearized in massively parallel nanochannels, excited with the appropriate lasers for backbone and labels detection, and optically imaged to reveal the pattern of labels on DNA molecules (FIG. 5A). Mapping to a reference genome, and basic metrics covering the center of mass of interrogated molecules, False Positive (FP) and False Negative (FN) were carried out using nanoStudio data analysis software (BioNano Genomics). Results are shown in FIG. 5B. Thus, nucleic acids can be purified and labeled in thin-layer porous matrices and labeled in accordance with some embodiments herein.

### Example 2

### Thin Layer-Based DNA Purification in a Tethered Well Followed by in-Matrix One Color Labeling

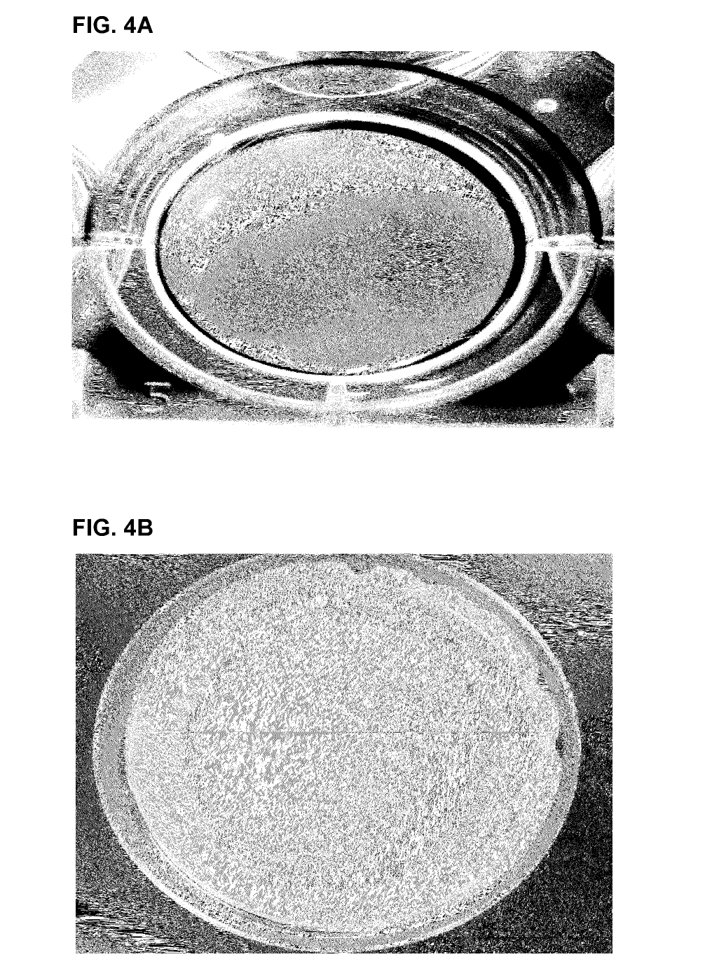

E. coli cells were mixed with an agarose solution and manually spread with a pipet tip on the surface of a well of a six-well plate to generate a thin layer. Upon solidification of the agarose-E. coli matrix at 4° C., a nylon mesh was place on top of the thin layer to keep it tethered to the surface (FIG. 4A) during the subsequent processing steps. The tethered agarose-E. coli layer was treated with lysozyme and proteinase K followed by several washes to remove contaminants, leaving clean DNA behind. DNA retained in the thin layer was nicked with Nt.BspQI, washed and labeled by nick translation with taq polymerase in the presence of green fluorescent dye coupled to dUTP. Following another wash step, the labeled nicks were repaired by treating with PreCR (New England BioLabs). The thin layer containing DNA was liquefied by melting the agarose and treating with agarase to liberate the DNA which was stained with YOYO and processed on the Irys™ system (BioNano Genomics) as outlined in example 1. Results are shown in FIG. 6. Thus, nucleic acids can be purified and labeled in thin-layer porous matrices in accordance with some embodiments herein.

### Example 3

### Microlayer-Based DNA Purification on a Slide/Thin Layer DNA Purification in a Well Followed by DNA Recovery and One Color Labeling in Solution

20 ul E. coli-agarose mixture was spread on a glass slide or well as described in Examples 1 and 2. The attached layer was treated with lysozyme and proteinase K followed by several washes to remove contaminants, leaving clean DNA behind. The agarose-DNA complex was liquefied by melting the agarose and treating with agarase. Following drop dialysis, the purified DNA was nicked with Nt.BspQI and labeled by nick translation with taq polymerase in the presence of green fluorescent dye coupled to dUTP. The labeled nicks were repaired by treating with PreCR (New England BioLabs). The resulting DNA was stained with YOYO and processed on the Irys™ system (BioNano Genomics) as described in Example 1. Results are shown in FIG. 7. Thus, nucleic acids can be purified in thin-layer porous matrices and labeled in accordance with some embodiments herein.

### Example 4

### Thin Layer-Based DNA Purification in a Tethered Plate Followed by DNA Recovery and One Color Labeling in Solution—Larger Scale

A 900 ul E. coli-agarose mixture was manually spread with a pipet tip on the bottom of a 10 cm culture plate. Upon solidification at 4° C., a nylon mesh was place on top of the thin layer attached to the bottom of the plate to keep it tethered to the plate's surface (FIG. 4B) during subsequent processing. The tethered agarose-E. coli thin layer was treated with lysozyme and proteinase K followed by several washes to remove contaminants, leaving clean DNA behind. The agarose-DNA complex was liquefied by melting the agarose and treating with agarase. Following drop dialysis, the purified DNA was nicked with Nt.BspQI and labeled by nick translation with taq polymerase in the presence of green fluorescent dye coupled to dUTP. The labeled nicks were repaired by treating with PreCR (New England BioLabs). The resulting DNA was stained with YOYO I and processed on the Irys™ system (BioNano Genomics) as described in example I. Results are shown in FIG. 8. Thus, nucleic acids can be purified in thin-layer porous matrices and labeled in accordance with some embodiments herein.

### Example 5

### Plug-Based DNA Purification Followed by Fragmentation and One Color Labeling in Porous Units

E. coli-agarose plugs were generated as described by BioRad (CHEF Bacterial Genomic DNA Plug Kit #170-3592). A plug containing bacterial cells was treated with lysosyme, followed by proteinase K and RNase leaving clean DNA behind. The plug was shred into small pieces by homogenizing in a microfuge tube with a blue pestle (Sigma). DNA retained in the porous units was nicked with Nt.BspQI, washed and labeled by nick translation with taq polymerase in the presence of green fluorescent dye coupled to dUTP. Following another wash step, the labeled nicks were repaired by treating with PreCR (New England BioLabs). After each wash the porous units were spun to concentrate at the bottom of the tube. The labeled DNA in porous units was liquefied by melting the agarose and treating with agarase. Following drop dialysis, the DNA was stained with YOYO I and processed on the Irys™ system (BioNano Genomics) as described in Example 1. Results are shown in FIG. 9. Thus, nucleic acids can be purified and labeled in porous units in accordance with some embodiments herein.

### Example 6

### Microlayer-Based DNA Purification Followed by in-Matrix One Color Labeling in Device of FIG. 8

E. coli cells were mixed with an agarose solution and spread in a 100 μm thick well on a glass slide defined by PTFE coating, by sandwiching the agarose-cell mixture with a non-stick slide. Upon solidification of the agarose-E. coli matrix at 4° C., the non-stick slide was removed leaving a 100 μm thick microlayer occupying the well of the PTFE coated slide (FIG. 3B). The slide was assembled into the processing device described in FIG. 10D. Proteinase K digestion in the reaction well was followed by several washes to remove contaminants, leaving clean DNA trapped in the microlayer. Labeling and repair were also carried out in the processing device. DNA trapped in the microlayer was nicked with Nt.BspQI, washed and labeled by nick translation with taq polymerase in the presence of green fluorescent dye coupled to dUTP. Following another wash step, the labeled nicks were repaired by treating with PreCR (New England BioLabs). The slide was removed from the device shown in FIG. 10D. The microlayer-containing DNA was harvested into a microfuge tube and liquefied by melting the agarose and treating with agarase to liberate the DNA which was stained with YOYO and processed on the Irys™ system (BioNano Genomics) as described in Example 1. Results are shown in FIG. 11. Thus, nucleic acids can be purified and labeled in thin-layer porous matrices in accordance with some embodiments herein.

### Example 7

### De Novo Assembly and Mapping

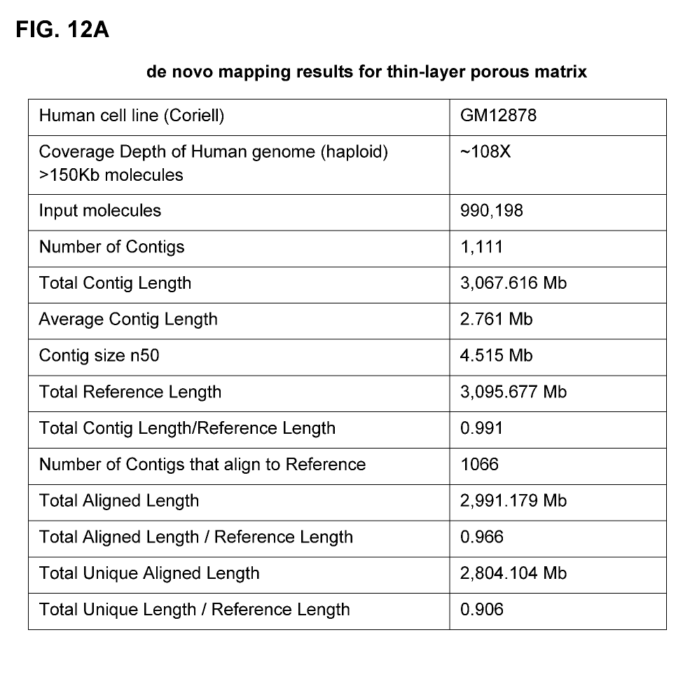

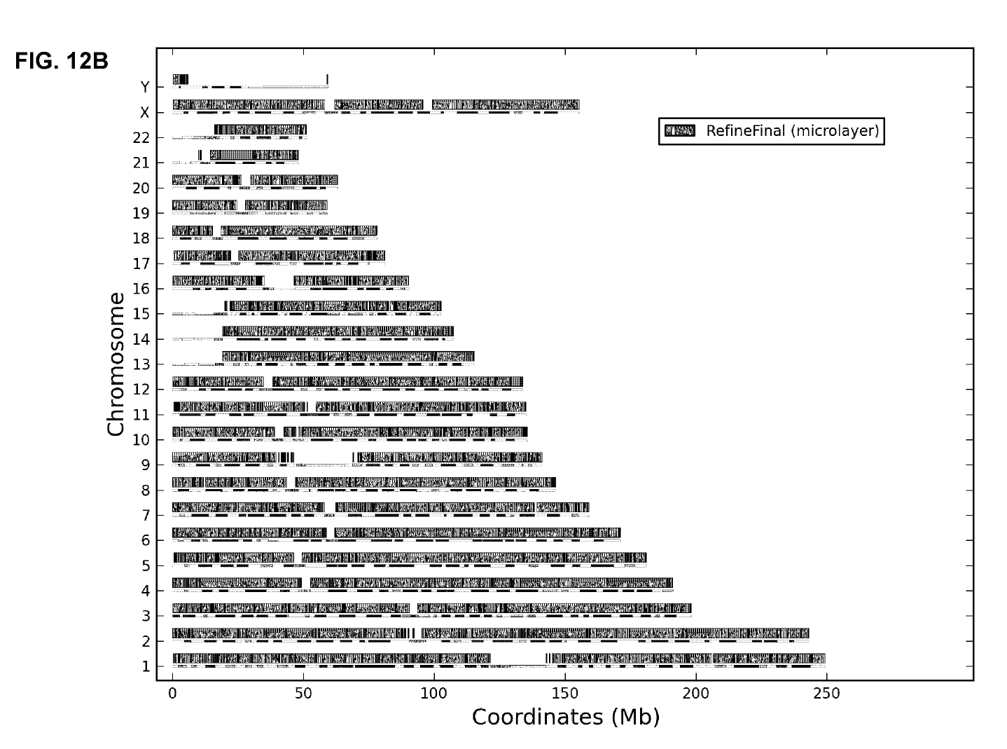

Cells from a human cell line (Coriell, catalog ID GM12878) were provided. The cells were mixed with an agarose solution and spread on a PTFE-coated glass slide to form a thin-layer porous matrix comprising the cells. The slide with and thin-layer porous matrix were positioned in the gap 25 of the system depicted in FIGS. 13A-13C, so that the thin-layer porous matrix could be heated by the system, accessed via a reaction well 27. Proteinase K digestion in the reaction well 27 was followed by several washes to remove contaminants, leaving clean DNA trapped in the thin-layer porous matrix. The DNA was labeled while in the thin-layer porous matrix: DNA was nicked with Nt.BspQI and labeled by nick translation with taq polymerase in the presence of green fluorescent dye coupled to dUTP. The labeled nicks were repaired by treating with PreCR (New England BioLabs) or as described in the IrysPrep™ Labeling—NLRS protocol (BioNano Genomics). The porous matrix comprising labeled DNA was harvested into a microfuge tube and liquefied by melting the agarose and treating with agarase to liberate the DNA which was stained with YOYO and linearized on the Irys™ system (BioNano Genomics) as described in Example 1. Labeled DNA molecules were aligned in an iterative fashion to generate contigs representing a de novo assembled human genome map. The de novo-generated contigs were aligned to the reference human map (HG19) and graphical depicted in FIG. 12B. Numeric metrics are displayed in FIG. 12A. Thus, thin-layer porous matrices and processing systems as described herein can be used to reliably isolate and label nucleic acids for analysis in a fluidic nanochannel system.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1.-179. (canceled)

180. A method of processing a sample comprising a polynucleotide, the method comprising:
immobilizing the sample in a thin-layer porous matrix;
conforming the thin-layer porous matrix to a substrate;
removing non-polynucleotide molecules from the thin-layer porous matrix conformed to the substrate while the polynucleotide remains immobilized in the thin-layer porous matrix; and
at least one of:
(a) labeling the polynucleotide with a first label; or
(b) separating the polynucleotide from the thin-layer porous matrix.

181. The method of claim 180, wherein the polynucleotide is labeled with the first label while immobilized in the thin-layer porous matrix and subsequently separated from the thin-layer porous matrix.

182. The method of claim 180, wherein the polynucleotide is separated from the thin-layer porous matrix, and subsequently labeled with the first label.

183. The method of claim 180, wherein immobilizing the sample in the thin-layer porous matrix comprises contacting the sample with a precursor of the thin-layer porous matrix, and forming the thin-layer porous matrix from the precursor comprising the sample.

184. The method of claim 180, wherein the sample is immobilized in the thin-layer porous matrix after the thin-layer porous matrix has been formed from a precursor of the thin-layer porous matrix.

185. The method of claim 180, wherein the thin-layer porous matrix is conformed to the substrate between the substrate and another entity, thereby defining at least one of a thickness, diameter, or volume of the thin-layer porous matrix.

186. The method of claim 180, wherein the thin-layer porous matrix has a thickness of about 1 to 999 micrometers.

187. The method of claim 180, wherein the thin-layer porous matrix comprises an agarose matrix, a polyacrylamide matrix, a gelatin matrix, a collagen matrix, a fibrin matrix, a chitosan matrix, an alginate matrix, a hyaluronic acid matrix, or any combination thereof.

188. The method of claim 180, wherein removing non-polynucleotide molecules from the thin-layer porous matrix or porous matrix is performed in the absence of an electric field.

189. The method of claim 180, wherein removing non-polynucleotide molecules from the thin-layer porous matrix or porous matrix does not comprise electrophoresis.

190. The method of claim 180, further comprising forming the thin-layer porous matrix by cooling a matrix precursor liquid in a mold.

191. The method of claim 180, further comprising forming the thin-layer porous matrix by cooling a matrix precursor, wherein the substrate defines at least one surface of the thin-layer porous matrix while the matrix percursor liquid cools.

192. The method of claim 180, further comprising:
forming the thin-layer porous matrix by placing a precursor over a surface other than the substrate; and
moving the precursor to the substrate after the precursor cools.

193. The method of claim 180, wherein after the thin-layer porous matrix has been formed, the sample is added to the thin-layer porous matrix by an electromagnetic field.

194. The method of claim 180, wherein the thin-layer porous matrix comprises agarose and has a thickness of 100 μm to 600 μm.

195. The method of claim 180, wherein immobilizing the sample in a porous matrix comprises adding the sample to the thin-layer porous matrix by an electromagnetic field.

196. The method of claim 180, wherein the substrate comprises a mesh with openings of 0.1 to 10 nm in diameter.

197. The method of claim 180, wherein separating comprises electroelution.

198. A method of processing a sample comprising a polynucleotide, the method comprising:
immobilizing the sample in a porous matrix, in an aqueous environment;
fragmenting the porous matrix comprising the immobilized sample;
removing non-polynucleotide molecules from the porous matrix while the polynucleotide remains in the porous matrix; and
separating the polynucleotide from the porous matrix.

199. A polynucleotide preparation comprising:
a thin-layer porous matrix conformed to a substrate;
a polynucleotide immobilized in the porous matrix, wherein the polynucleotide is substantially isolated from non-polynucleotide cellular components, and wherein the polynucleotide has been site-specifically labeled or enzymatically modified while in the matrix.
